(19) United States
(12) Patent Application Publication
(10) Pub. No.: US 2023/0303999 A1
(43) Pub. Date: Sep. 28, 2023

(54) COMBINATORIAL PLATFORM FOR HIGH-THROUGHPUT POLYNUCLEOTIDE-ENCODED CATALYST DISCOVERY

(71) Applicant: WISCONSIN ALUMNI RESEARCH FOUNDATION, Madison, WI (US)

(72) Inventors: Jeffrey Martell, Madison, WI (US); Edward Pimentel, Madison, WI (US); Justice Merrifield, Madison, WI (US)

(21) Appl. No.: 18/189,056

(22) Filed: Mar. 23, 2023

Related U.S. Application Data
(60) Provisional application No. 63/322,948, filed on Mar. 23, 2022.

(51) Int. Cl. C12N 15/10

(52) U.S. Cl. CPC C12N 15/1065

(57) ABSTRACT

The present disclosure provides a polynucleotide scaffold platform for development and screening of catalytic libraries. The platform and methods can be used for screening for new synthetic catalysts and for improving catalytic reactions in a high-throughput manner.

### CROSS-REFERENCE TO RELATED APPLICATIONS

The present application claims priority to U.S. Provisional Patent Application No. 63/322,948 filed Mar. 23, 2022, the entire contents of which are hereby incorporated by reference.

### STATEMENT REGARDING FEDERALLY SPONSORED RESEARCH

N/A

### FIELD OF THE INVENTION

The disclosed technology is generally directed to catalyst discovery. More particularly the technology is directed to a combinatorial platform for high-throughput polynucleotide-encoded catalyst discovery.

### BACKGROUND OF THE INVENTION

Catalysts play countless roles in modern society, including production of fertilizer that supports the human population; synthesis of plastics and other ubiquitous materials; and development of pharmaceuticals that treat diseases. Despite the marvels of existing catalysts, many of them require harmful reaction solvents or high reaction temperatures, which cost energy. For example, the Haber-Bosch process for producing ammonia consumes ˜1-2% of the world's energy supply. Because of the global climate crisis, there is an urgent need to develop catalysts that operate under environmentally benign conditions.

Remarkably, natural enzymes have evolved to catalyze many reactions of great societal importance under mild conditions. For example, nitrogenase enzymes produce ammonia in aqueous solution at ambient temperatures, unlike the energy intensive Haber-Bosch process. Natural enzymes also catalyze reactions of relevance for therapeutics, diagnostics, energy conversion, bioremediation, and chemical synthesis. Enzymes achieve these impressive catalytic properties through the precise pre-organization of multiple functional groups in a flexible cavity, called an “active site,” leading to rate accelerations as large as 10¹⁷ relative to uncatalyzed reactions. Despite their remarkable properties, enzymes cannot catalyze as broad a scope of reactions as synthetic catalysts because their building blocks are limited to natural amino acids and cofactors.

Thus, conventional catalyst discovery involves low throughput testing of substrates, catalysts, co-catalysts, and additives. There is a need in the art for high-throughput methods to develop and discover new active catalyst and further components for improved catalytic activity.

### BRIEF SUMMARY OF THE INVENTION

The present invention relates to a novel scaffold platform for the making and selecting of catalytic systems, catalytic libraries and methods of making and using the same. One aspect of the technology provides for a polynucleotide barcoded building block oligomer system for preparing a catalyst library system. The polynucleotide system comprises at least two sets of single stranded polynucleotides, each set of single stranded polynucleotides characterized by a catalytic component selected from a panel of catalytic components linked to single stranded polynucleotides of the set. Each single stranded polynucleotide of a set comprises a polynucleotide barcode indicative of the catalytic component selected from the panel of catalytic components linked to the single stranded polynucleotide, a domain complementary to a domain possessed by single stranded polynucleotides of a second set of single stranded polynucleotides and optionally a domain complementary to a domain possessed by single stranded polynucleotides of a third set of single stranded polynucleotides. Each single stranded polynucleotide of one set is capable of hybridizing with each single stranded polynucleotide of at least one other set to form a self-assembled polynucleotide nanoscaffold. The self-assembled polynucleotide nanoscaffold comprises a catalytic active site comprising the catalytic components and a barcode signature indicative of the catalytic active site. One set of single stranded polynucleotides may comprise a catalytic component selected from a panel of catalysts or catalyst binding ligands and another set of single stranded polynucleotides comprises a catalytic component selected from a panel of substrates. In some embodiments, the system comprises 3, 4, or 5 sets of single stranded polynucleotides.

Another aspect of the technology provides for a catalyst system library. The catalyst system library comprises a plurality of self-assembled polynucleotide nanoscaffolds. Such self-assembled polynucleotide nanoscaffolds may be prepared from any of the polynucleotide barcoded building block oligomer systems described herein. The self-assembled polynucleotide nanoscaffolds may comprise a single stranded polynucleotide selected from each set of single stranded polynucleotides of the polynucleotide barcoded building block oligomer system.

Another aspect of the technology provide for methods for assembling a catalyst system library. The method comprises preparing a polynucleotide barcoded building block oligomer system, wherein a set of single stranded polynucleotides is prepared by
(i) distributing a first single stranded polynucleotide comprising a domain complementary to a domain possessed by a second single stranded polynucleotide and optionally a domain complementary to a domain possessed by a third stranded polynucleotide between a set of containers,
(ii) adding to each container of the set of containers a catalytic component selected from a panel of catalytic components and a polynucleotide barcode indicative of the catalytic component selected from the panel,
(iii) attaching to the single stranded polynucleotide the catalytic component selected from a panel of catalytic components, and
(iv) ligating the single stranded polynucleotide and the polynucleotide barcode.
   The method further comprises combining the two or more sets of single stranded polynucleotides under conditions sufficient to prepare self-assembled polynucleotide nanoscaffolds; and optionally ligating the self-assembled polynucleotide nanoscaffolds to prepare a continuous polynucleotide sequence.

Another aspect of the technology provides for a method of identifying catalytic activity. The method comprises exposing any of the catalyst system libraries described herein to catalytic reaction conditions and identifying self-assembled polynucleotide nanoscaffolds that react under the catalytic reaction conditions. Identifying self-assembled polynucleotide nanoscaffolds that react under the catalytic reaction conditions may comprise isolating self-assembled polynucleotide nanoscaffolds with catalytic activity, amplifying a portion of the self-assembled polynucleotide nanoscaffolds comprising the barcode signature, and sequencing the portion of the self-assembled polynucleotide nanoscaffolds to determine the barcode signature.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1: A schematic showing exemplary polynucleotide barcoded building block oligomer system, including sets of single stranded polynucleotides, 100, 200, 300, including catalytic components, 141, 142, 14L, 241, 242, 24M, 341, 342, 34N, selected from a panel of catalytic components linked to single stranded polynucleotides. Each single stranded polynucleotide of a set comprises a polynucleotide barcode, 161, 162, 16L, 261, 262, 26M, 361, 362, 36N, indicative of the catalytic component selected from the panel of catalytic components linked to the single stranded polynucleotide. In these exemplary schematics, pairs of complementary domains, 118 and 228, 119 and 339, are also shown. The complementary domains of each single stranded polynucleotide of one set are capable of hybridizing with each single stranded polynucleotide of at least one other set to form self-assembled polynucleotide nanoscaffolds.

FIG. 2: A schematic showing a plurality of self-assembled polynucleotide nanoscaffolds 400, including an index of individual nanoscaffolds.

FIG. 3: A schematic showing one polynucleotide nanoscaffold 521 that has been ligated between a first single stranded polynucleotide 121 and a second single stranded polynucleotide 221 and the first single stranded polynucleotide 121 and a third single stranded polynucleotide 331.

FIG. 4: A schematic overview of the combinatorial platform to screen 10⁸ supramolecular catalysts.

FIG. 5: A schematic showing the structure of an assembled polynucleotide nano-scaffold. Shown in the catalytic active site are catalytic components, including a substrate, X, a catalyst, M, an additive, A, and a base, B. Each single stranded polynucleotide is outfitted with a polynucleotide bar code corresponding to each of the catalytic components. Y represents a reactant.

FIG. 6: A schematic showing how 10⁸ unique polynucleotide nano-scaffolds can be generated in a single test tube for high-throughput catalyst screening.

FIG. 7: A schematic of the procedure for assembling 10⁸ DNA-barcoded candidate catalysts.

FIG. 8: Procedure for selecting and identifying high-activity catalysts from a library of 10⁸ DNA nano-scaffolds. Panel A shows the polynucleotide nano-scaffold library that is surveyed for self-oxidation of an alcohol by an active catalytic combination to an aldehyde or ketone. In a second step, the polynucleotide nanoscaffold library is exposed to reductive amination conditions where the successfully oxidized substrates are tagged with biotin. Panel B shows how streptavidin pulled down can be used to isolate the successful, biotin-tagged polynucleotide nanoscaffolds from the library mixture and can be amplified by PCR. In a final step, the polynucleotide nanoscaffolds can be sequenced and the barcodes corresponding to catalytic components read out.

FIG. 9: The ability of promising catalytic combinations to oxidize a free-floating substrate (not tethered) will be evaluated. Importantly, either outcome—if the polynucleotide scaffold is essential for activity or if reactivity can be transferred off the polynucleotide scaffold—are positive outcomes for catalytic discovery.

FIG. 10: A schematic showing a polynucleotide nanoscaffold for bimetallic catalyst discovery, including the possible variants for achieving a greater than 10⁶ possible combinations tested in a single test tube.

FIG. 11: A schematic showing how to use a palladium catalyst polynucleotide nanoscaffold library.

FIG. 12: A schematic showing how to make a palladium catalyst polynucleotide nanoscaffold library with a panel of four palladium catalysts (C1-C4) with corresponding unique barcodes and a panel of forty boronic acid substrates (B1-B40), to achieve a quantitative ranking of 160 catalyst-substrate pairs.

FIG. 13: A schematic showing a selection of catalytic components for developing a polynucleotide nanoscaffold library for debenzylation catalyst discovery. Panel a) shows a schematic of a library composition. Panel b) shows biotin removal selection scheme where the successful catalytic combinations lack the biotin affinity tag.

FIG. 14: A schematic showing a selection of catalytic components for developing a polynucleotide nanoscaffold library for nickel-photoredox catalyst discovery. Panel a) shows a schematic of a library composition. Panel b) shows biotin removal selection scheme where the successful catalytic combinations are tagged with biotin.

FIG. 15: A schematic showing an approach to generate a polynucleotide nanoscaffold library for the discovery of catalysts to degrade chemical warfare agents, including the preparation of a masked biotin for bioconjugation to the catalyst active site for downstream isolation of successful catalytic combinations.

FIG. 16: A model of a polynucleotide nanoscaffold with a detailed view of the catalytic active site (inset).

FIG. 17: A schematic illustration of a three-component polynucleotide nanoscaffold architecture for combinatorial catalytic screening. Barcoding regions of the polynucleotide sequence are correlated to each unique catalytic component in the assembled nanoscaffold. The two larger hairpins on the left and right are the primer binding sites for amplification.

FIG. 18: A method for bioconjugation of catalytic components to polynucleotides. Panel A shows a synthetic scheme for bioconjugation. Panel B shows a high-performance liquid chromatogram and panel C shows an electrospray ionization mass spectra confirming the synthetic method. This method has been demonstrated with additional ligands including proline, alternative bipyridine ligands, various alcohol substrates, alkynes, phosphines, aromatic and alkyl groups, and organometallic complexes.

FIG. 19: A schematic of successful barcode attachment for identifying individual catalytic components. The attachment of hairpin barcodes proceeds quantitatively. In the gel shown there was a slight excess of barcoding hairpin.

FIG. 20: An electrophoretic gel showing successful oxidation and reductive amination to attach biotin for isolating successful catalytic combinations. The gel shows that biotin attachment to an aldehyde leads to a gel shift. When all the reaction components are present, oxidation if the alcohol creates an aldehyde in situ and biotin-PEG7-NH2 can be attached through reductive amination. When one or more components are missing, no shift is observed (not shown).

FIG. 21: Electrophoretic gels showing the assembly and ligation of polynucleotide nanoscaffolds. High-yielding assembly is observed in the native gel and is also demonstrated with DNA oligo with catalytic components attached. Denaturing gels shows that the nanostructure is successfully ligated. Downstream PCR is also successful.

FIG. 22: Streptavidin pull-down can be used to enrich biotin-tagged structures. Mixtures of polynucleotide structures with and without biotin (with barcodes to indicate the presence or absence of the biotin modification) were exposed to magnetic streptavidin beads. Biotinylated structures bound tightly, while unmodified structures were washed away. Amplification of the sequences before and after pull-down followed by sequencing shows that only the biotin barcode is identified after pull-down.

FIG. 23: The molecule used in polymerase chain reaction (PCR) amplification as an internal attachment handle. It is tolerated by Taq polymerases. Successful amplification of polynucleotide nanoscaffolds with multiple reaction components has been demonstrated.

FIG. 24: Successful Application of the Combinatorial Catalyst Platform to a Model 8-Member Library. Next-generation DNA sequencing reads of an 8-member barcoded model library. This proof-of-principle library was exposed to copper to allow catalytically competent library members to oxidize the DNA-linked alcohol substrates, followed by biocytin-hydrazide labeling of aldehydes generated during the oxidation step and a magnetic streptavidin bead pull-down to isolate the successfully oxidized and biotinylated library members. Both naive library (not exposed to reaction conditions) and the library members bound to the beads were amplified by PCR and submitted for next-generation DNA sequencing. The bar chart above shows the number of reads for each unique combination of DNA barcodes in the naive and the selected library samples. The sample containing each reaction component (bpy ligand, TEMPO, and alcohol substrate) is enriched relative to the library, demonstrating that the combinatorial catalyst discovery platform is capable of enriching the barcode frequency of a successful library member in the next-generation sequencing readout. The second-most enriched set of barcodes corresponds to the ligand-free library member. Because the bpy ligand is accelerating but not essential for the alcohol oxidation reaction, this demonstrates that the combinatorial catalyst discovery platform can also discriminate between excellent and poor catalysts.

FIG. 25: Preparation of a barcoded, 96-member library of ligands, co-catalysts and substrates for alcohol oxidation. To demonstrate that the catalyst discovery platform can be scaled to library sizes larger than the 8-member proof-of-principle library used for platform rehearsal in FIG. 24, a barcoded 96-member library was prepared with 4 different components at the ligand position, 4 different components at the co-catalyst position, and 6 different components at the substrate position.

FIG. 26: Using the Combinatorial Catalyst Platform for Photocatalytic C—H Arylation Reactions. To demonstrate the viability of the platform for screening photocatalytic reactions, a photochemical C—H arylation reaction demonstrated on DNA by Molander (Krumb et al. Chem. Sci. 2021, 13, 1023) and demonstrated it on DNA (A) with a biotinylated coupling partner (the scope of the coupling partner could be much wider) using a variety of photocatalysts (free-floating fluorescein, Ru(bpy)₃Cl₂, Ir(ppy)₃, Ir(dtbbpy)(dF(CF₃)ppy)₂PF₆ etc. all work—the key parameter is that the photocatalyst reduction potential is sufficient to reduce the aryl iodide). To demonstrate that the viability of the strategy, an initial simplified architecture with barcoded hairpin loops that act as primer binding sites for PCR (C) was created, and then demonstrated with individually annealed architectures that only architectures bearing both the substrate and the catalyst lead to self-biotinylation (validated by streptavidin gel shift assay, above in D).

FIG. 27: 8-member proof-of-principle library. To demonstrate that the platform can enrich a nanostructure that contains two catalysts and a substrate, a model 8-member barcoded library was prepared with two variants at each position of the nanostructure, either bearing or lacking a ligand, a co-catalyst, or a substrate. This library was then subjected to oxidation and aldehyde-labelling conditions, affinity pull-down, PCR amplification and sequencing.

### DETAILED DESCRIPTION OF THE INVENTION

A fundamental challenge that has eluded chemists for decades is the creation of synthetic catalysts that mimic the ability of natural enzymes to carry out rapid and selective transformations under mild conditions. The present disclosure combines synthetic catalysis, polynucleotide nanomaterials, and next-generation sequencing to create a new platform for the rapid discovery of enzyme-mimicking catalysts. This new system dramatically accelerates the discovery of catalysts with diverse potential applications and the elucidation of fundamental catalytic mechanisms.

### Polynucleotide Nanoscaffold Systems and Libraries

The present disclosure describes a polynucleotide scaffold platform in which a large number (e.g. 10⁴-10¹² or more) supramolecular catalysts, each displaying distinct abiotic groups that are capable of interacting, can be synthesized in a single test tube and then tested. Thus high-throughput one pot screening of catalysts can be performed using the scaffold platform and methods described herein. These methods can be used to identify new novel active catalysts and catalytic mixtures. In one embodiment, the current disclosure creates enzyme-mimicking synthetic molecules consisting of active sites that pre-organize abiotic functional groups. Such molecules would combine the sophistication of enzyme active sites with the enhanced reactivity of abiotic chemistry, unlocking fundamentally new catalytic mechanisms.

Previous enzyme-mimicking catalysts have generally been orders of magnitude less efficient than natural enzymes because it is exceedingly difficult to replicate the complex chemical environment of enzymes. The primary challenges are: 1) enzymes possess irregular shapes that are difficult to mimic in synthetic molecules, 2) enzyme active sites contain multiple precisely-placed functional groups, which are difficult to install into synthetic cage-like molecules, and 3) whereas enzymes are optimized through evolution, synthetic catalysts are developed by low-throughput synthesis and testing.

The present disclosure overcomes these difficulties to provide synthetic catalyst and catalytic systems which exhibit at least some of the following attributes: (a) three-dimensional active site that surrounds a catalytic metal and the reaction substrates, (b) multiple functional groups that are precisely placed close to the catalytic metal, (c) combinatorial synthesis and testing of millions of active sites to mimic natural evolution, (d), conformational flexibility to avoid inhibition by the reaction product.

Supramolecular synthetic catalysts that mimic enzymes combines the selectivity of enzymes with the expanded reactivity of synthetic catalysts. Creating such enzyme-mimicking catalysts required architectures in which multiple reactive groups are displayed into a three-dimensional structure. Polynucleotides provided the building material for the creation of such supramolecular enzyme mimicking catalysts because of the following advantages: (i) predictable self-assembly of 3D nanostructures featuring cavities with diverse sizes and shapes, (ii) diverse abiotic groups can be attached site-specifically on supramolecular DNA/RNA architectures, (iii) the DNA/RNA encodes information (e.g., polynucleotide barcodes) and thus enables high-throughput combinatorial reaction discovery, (iv) DNA is chiral and can be exploited for stereo- and regio-selective reactions, and/or (v) process called “SELEX” can be used to discover small molecule-binding DNA structures called “aptamers,” which can be exploited for substrate binding in catalysis.

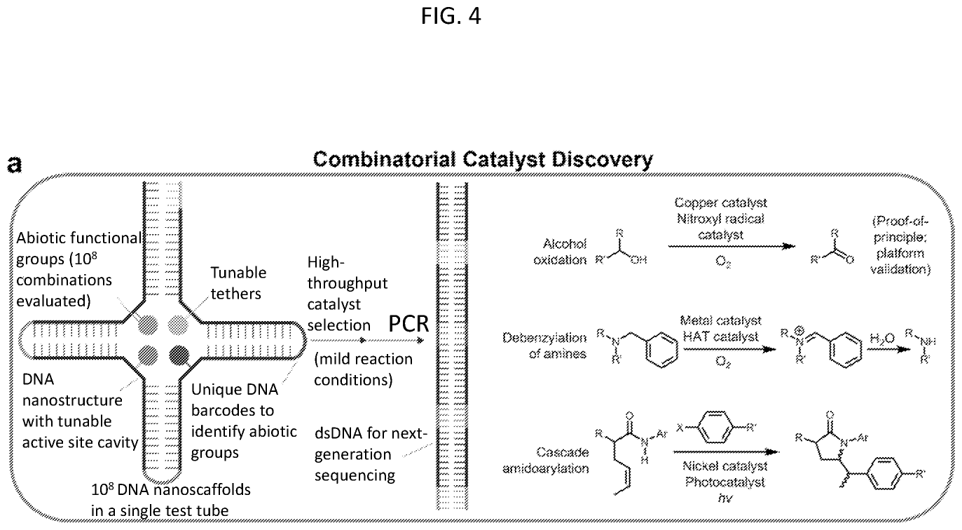

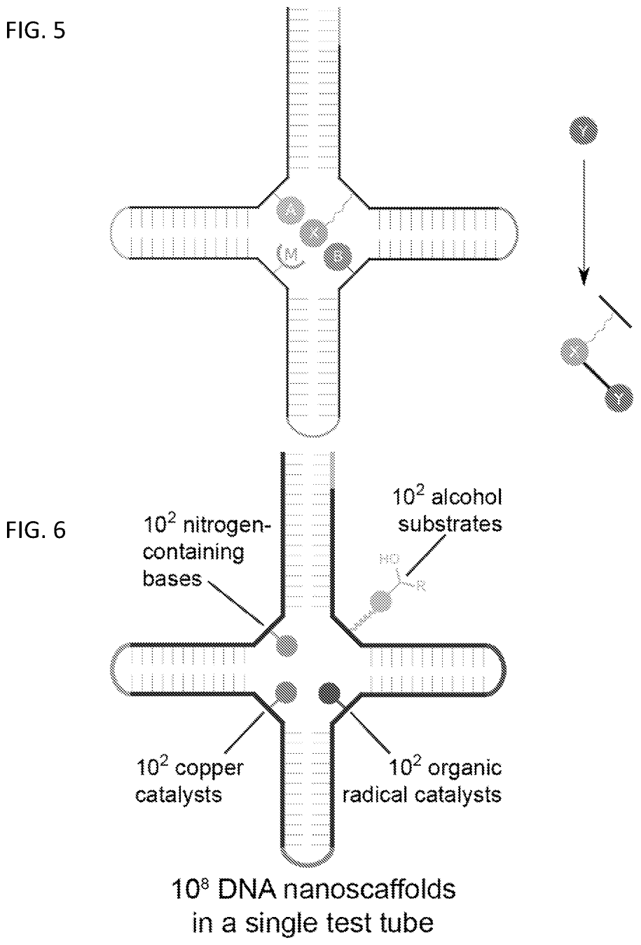

The present disclosure provides novel enzyme-mimicking catalysts fulfilling these attributes described herein using polynucleotide nanoscaffolds (e.g., DNA nano-scaffolds or RNA nano-scaffolds), combining DNA/RNA nanotechnology, DNA/RNA-compatible synthetic chemistry, and next-generation DNA sequencing (FIG. 4).

The present polynucleotide nanoscaffold platform is different from previous approaches because it combines multiple disciplines that are under-utilized in catalyst development. First, advances in DNA nanomaterials allows flexible three-dimensional cavities and structures to be created with tunable sizes and shapes. Second, DNA-compatible synthetic chemistry allows multiple (e.g., ≥4) abiotic functional groups to be displayed in precise locations on the DNA scaffold to place them in close proximity to each other. Third, large libraries of candidate catalysts (e.g., ≥10⁸) can be synthesized in a single test tube using combinatorial self-assembly. Fourth, DNA barcoding, high-throughput catalyst selection, and DNA sequencing allows rapid identification of the most active catalysts from the libraries, thus mimicking natural evolution. This platform offers the unprecedented capability to rapidly measure the outcome of millions of catalytic reactions in a single test tube, taking advantage of DNA sequencing technology that has revolutionized biology and holds tremendous untapped potential to facilitate discovery in synthetic catalysis.

The present disclosure provides methods, catalyst systems, and catalyst system libraries using self-assembled polynucleotide nanoscaffolds comprising catalytic components that can be used to screen for and identify active synthetic catalysts.

In one embodiment, a catalyst system library is provided. The catalyst system library comprises a plurality of self-assembled polynucleotide nanoscaffolds. The catalyst system library can be made by the methods described herein and can then be used to test and screen for catalytic activity, identifying the combination of elements that provide catalytic activity or enhancing catalytic activity.

In some embodiments, the plurality of self-assembled polynucleotide nanoscaffolds each form a distinct catalytic active site (e.g., cavity) comprising a combination of different catalytic components that can be screened and identified (as depicted in FIG. 4 using a 4-mer, however, 3-mer, 4-mer, 5-mer, 6-mer, and other structures are contemplated). In other embodiments, the self-assembled polynucleotide nanoscaffolds use their three-dimensional structure to bring the catalytic components into close proximity, as described more herein.

In some embodiments, the polynucleotide nanoscaffold comprises or consists of three or more catalytic components and at least three complementary polynucleotide sequences that bring the catalytic components in sufficiently close proximity for a catalytic reaction to occur—herein called “3-mers”. In another embodiment, the polynucleotide nanoscaffold comprises or consists of four or more catalytic components and at least four complementary polynucleotide sequences that bring the catalytic components in sufficiently close proximity for a catalytic reaction to occur, herein called “4-mers”. In the same logic, 5-mers, 6-mers, 7-mers, and onwards to 100-mers or more could be made. In some embodiments the catalytic library comprises of polynucleotide nanoscaffolds with a mixture of nanoscaffold sizes, for example a mixture of 3-mers and 4-mers, in a single test tube. In other embodiments there could be a mixture of at least three or more nanoscaffold sizes. In some embodiments, the catalyst system library may comprise a mixture of 3-mer, 4-mer, 5-mer etc., depending on the catalyst to be tested, and allows for the ability to test different combinations of components to increase the catalytic activity (e.g., adding different number of components may allow in some embodiments the ability to fine-tune the activity of the catalysts by finding 3-component and 4-components using the same catalyst but having different reaction times or different activity).

In some embodiments, each polynucleotide nanoscaffold comprises (i) a first catalytic component linked to first single stranded polynucleotide comprising a first polynucleotide barcode; (ii) a second catalytic component linked to the second single stranded polynucleotide comprising a second polynucleotide barcode, and at least (iii) a third catalytic component linked to a third single stranded polynucleotide comprising a third polynucleotide barcode. The first, second and third single stranded polynucleotide each linked to a catalytic component self-assemble into a three-dimensional polynucleotide nanostructure (nanoscaffold) that forms a catalytic active site.

In some embodiments, the polynucleotide nanoscaffold further comprises (iv) a fourth catalytic component linked to a fourth single stranded polynucleotide comprising a fourth polynucleotide barcode, wherein the first, second, third and fourth single stranded polynucleotide self-assemble into the three-dimensional polynucleotide nanostructure. In another embodiment, the self-assembled polynucleotide nanoscaffolds further comprises (v) a fifth catalytic component linked to a fifth single stranded polynucleotide comprising a fifth polynucleotide barcode, wherein the first, second, third, fourth and fifth single stranded polynucleotide that self-assemble into the three-dimensional polynucleotide nanostructure. In yet a further embodiment, the self-assembled polynucleotide nanoscaffolds further comprises at least one additional catalytic component linked to at least one additional single stranded polynucleotide comprising an additional polynucleotide barcode. It is contemplated that additional catalytic components attached via additional ss polynucleotides to allow for variations in the number of the catalytic components capable of being into close proximity (e.g., 3, 4, 5, 6, 7, 8, 9, 10, 11, 12, 13, 14, 15, 20, etc.) and is only limited by the ability of the ss polynucleotides to self-assemble into three dimensional structures. The ability of each additional catalytic component to be tethered to an additional ss polynucleotide that has a unique barcode allows for the later identification of the different catalytic components to be determined that were complexed in close proximity with each other via the 3-D nanostructure.

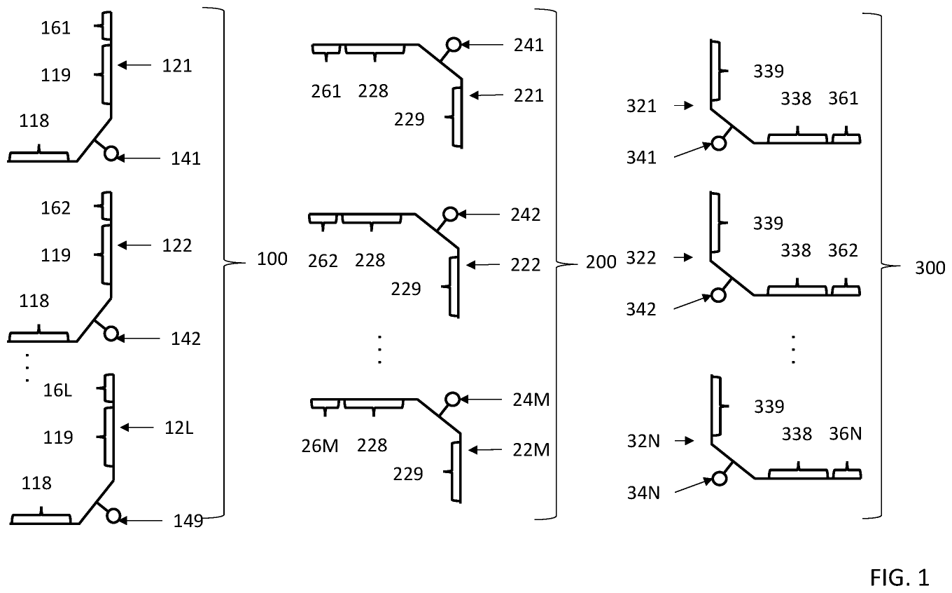

Referring now to FIG. 1 where an exemplary polynucleotide barcoded building block oligomer system for preparing a catalyst library system. By way of example, FIG. 1 shows a library of three sets of single stranded polynucleotides (100, 200, 300). This is readily extendible to four, five, six or higher number of sets of single stranded polynucleotides.

Each set of single stranded polynucleotides is characterized by a catalytic component selected from a panel of catalytic components which are linked to single stranded polynucleotides of the set. Each single stranded polynucleotide of a set also includes a polynucleotide barcode indicative of the catalytic component selected from the panel of catalytic components linked to the single stranded polynucleotide. Each single stranded polynucleotide of a set also includes a domain complementary to a domain possessed by single stranded polynucleotides of a second set of single stranded polynucleotides, and optionally a domain complementary to a domain possessed by single stranded polynucleotides of a third set of single stranded polynucleotides.

Each panel of catalytic components comprises a number of selectable catalytic components. A panel may have N selectable components, where N is an integer. Each panel may have an integer number of selectable components, e.g., L, M, and N for each of three panels. Panels may have the same number of selectable components, e.g., N=M, but need not. Each panel may have a unique number of selectable components, e.g., N≠M. In exemplary embodiments, each panel may independently have at least 2, 5, 10, 20, 30, 40, 50, 60, 70, 80, 90, 100, 150, 200, 250, 300, 350, 400, 450, 500, 600, 700, 800, 900, or 1000 selectable components. When sets of single stranded polynucleotides are combined to prepare a catalyst system library comprising a plurality of self-assembled polynucleotide nanoscaffolds, the plurality of self-assembled polynucleotide nanoscaffolds may be determined by the product of the number of selectable components for each panel. For example, if there are two, three, or four panels of selectable catalytic components, the plurality of self-assembled nanoscaffolds is N×M, N×M×L, N×M×L×K, respectively.

Using FIG. 1 as an example, the first set of single stranded polynucleotides (100) comprises first single stranded polynucleotides, 121, 122, . . . 12L, where L represents the Lth selectable catalytic component of a panel. The ellipsis in FIGS. 1-3 are used for brevity to indicate the omission of elements from the set that are readily understood to be present. The first single stranded polynucleotides, 121, 122, . . . 12L, includes a first catalytic component, 141, 142, . . . 14L, respectively, a polynucleotide barcode, 161, 162, . . . 16L, respectively, indicative of the catalytic component, 141, 142, . . . 14L, and a domain complementary 118 to a domain possessed by single stranded polynucleotides of a second set of single stranded polynucleotides 228, and optionally a domain complementary to a domain possessed by single stranded polynucleotides of a third set of single stranded polynucleotides 339.

Similarly, the second set of single stranded polynucleotides 200 comprises second single stranded polynucleotides, 221, 222, . . . 22M where M represents the Mth selectable catalytic component of a panel. The second single stranded polynucleotides, 221, 222, . . . 22M, includes a second catalytic component, 241, 242, . . . 24M, respectively, a polynucleotide barcode, 261, 262, . . . 26M, respective, indicative of the catalytic component, 241, 242, . . . 25M, and a domain complementary 228 to a domain possessed by single stranded polynucleotides of a second set of single stranded polynucleotides 118, and optionally a domain complementary to a domain possessed by single stranded polynucleotides of a third set of single stranded polynucleotides (not shown in FIG. 1).

The third set of single stranded polynucleotides 300 comprises third single stranded polynucleotides, 321, 322, . . . 32N where N represents the Nth selectable catalytic component of a panel. The third single stranded polynucleotides, 321, 322, . . . 32N, includes a third catalytic component, 341, 342, . . . 34N, respectively, a polynucleotide barcode, 361, 362, . . . 36N, respectively, indicative of the catalytic component, 341, 342, . . . 34N, and a domain complementary 339 to a domain possessed by single stranded polynucleotides of a second set of single stranded polynucleotides 119, and optionally a domain complementary to a domain possessed by single stranded polynucleotides of a third set of single stranded polynucleotides (not shown in FIG. 1).

The example disclosed in FIG. 1 is readily extendible to four, five, six or higher number of sets of single stranded polynucleotides.

Each single stranded polynucleotide of one set, 100, 200, or 300, is capable of hybridizing with each single stranded polynucleotide of at least one other set to form a self-assembled polynucleotide nanoscaffold. For example, a first complementary domain 119 of single stranded polynucleotide 121 may hybridize with a complementary domain 339 of single stranded polynucleotide 321. A second complementary domain 118 of single stranded polynucleotide 121 may hybridize with a complementary domain 228 of single stranded polynucleotide 221. This is readily extendible to four, five, six or higher number of sets of single stranded polynucleotides. For example, the second single stranded polynucleotide 221 may contain a second complementary domains 229 which may hybridize with a complementary domain of fourth, fifth, sixth or higher order single stranded polynucleotide sets.

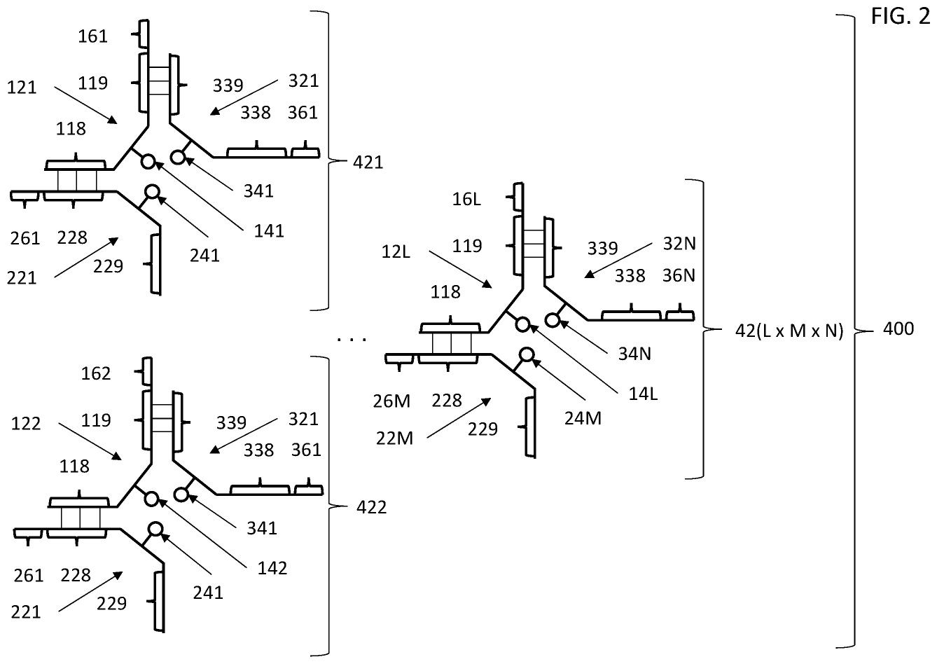

FIG. 2 illustrates the hybridization of complementary domains of the polynucleotides shown in FIG. 1. Hybridization of complementary domains self-assembles polynucleotides into a plurality of nanoscaffolds 400. The plurality of nanoscaffolds may be characterized by indexes of individual nanoscaffolds, 421, 422, and 42(L×M×N), that are the result of all the possible combinations for hybridization. The self-assembled nanoscaffold includes a catalytic active site including the catalytic components.

The nanoscaffolds of illustrated in FIG. 2 may be utilized in the catalyst screening methods described herein to identify catalytic components most useful for a reaction. In such a case, each nanoscaffold, e.g., 421, can also comprise primer binding sites on each polynucleotide, 121, 221, and 321, to allow for identification of the catalytic components, 141, 241, and 341, by amplifying barcodes 161, 261, and 361. The primer binding sites are not specifically shown but may be included as a portion of each barcode, 161, 261, and 361, or any other suitable portion of each polynucleotide 121, 221, and 321.

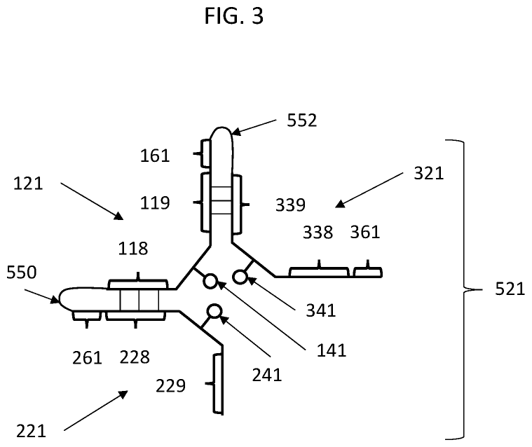

FIG. 3 provides an example of one nanoscaffold that has been ligated to make a polynucleotide sequence that may be read out as a barcode signature of the catalytic components present in the self-assembled nanoscaffold. The segments 550 and 552 join polynucleotides 121, 221, and 332. Nanoscaffold 521 comprises the catalytic components, 141, 241, 341, forming the catalytic active site. Nanoscaffold 521 also has a barcode signature indicative of the catalytic components present in the catalytic active site that includes polynucleotide barcodes 161, 261, and 361.

The nanoscaffolds of illustrated in FIG. 3 may be utilized in the catalyst screening methods described herein to identify combinations of catalytic components most useful for a reaction. In such a case, each nanoscaffold, e.g., 521, can also comprise primer binding sites on polynucleotides, 121 and 321, to allow for identification of the catalytic components, 141, 241, and 341, by amplifying barcodes 161, 261, and 361. The primer binding sites are not specifically shown but may be included as a portion of barcodes, 161 and 361, or any other suitable portion of polynucleotides 121 and 321.

The term “polynucleotide,” “oligonucleotide” or “nucleic acids” can be used interchangeably and refer to polymers comprising nucleotides or nucleotide analogs (e.g., a string of at least three or more) joined together through backbone linkages such as but not limited to phosphodiester bonds. The terms “nucleic acid” and “nucleic acid molecule,” as used herein, refer to a compound comprising a nucleobase and an acidic moiety, e.g., a nucleoside, a nucleotide, or a polymer of nucleotides. Polynucleotides include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. The terms “nucleic acid,” “DNA,” “RNA,” and/or similar terms include nucleic acid analogs, i.e. analogs having other than a phosphodiester backbone. Nucleic acids can be produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Although DNA is discussed as the preferred polynucleotide, including in the examples, it is to be understood that RNA or other polynucleotide structures are capable of being used in the same way as described for DNA nanostructures (nanoscaffolds).

Within each self-assembled polynucleotide nanoscaffold that makes up the library, each of the first, second, and at least third single stranded polynucleotide (e.g., DNA or RNA) comprises at least a portion complementary to the other single stranded polynucleotides (e.g., a portion of the first strand is complementary to a portion of the second and third in a 3-mer; a portion of the first strand is complementary to a portion of the second and fourth, a portion of the second strand is complementary to a portion of the first and third strand, and a portion of the third is complementary to a portion of the second and fourth strand in a 4-mer (FIG. 4), etc.). The complementary regions allow for the self-assembly of the polynucleotide nanoscaffold (e.g., DNA nanoscaffold or RNA nanoscaffold). In one embodiment the polynucleotide nanoscaffolds include complementary strands of deoxyribonucleic acid (DNA). In another embodiment, the polynucleotide nanoscaffolds include complementary strands of ribonucleic acid (RNA).

The term “catalytic component” as used herein refers to any component necessary in the reaction for a catalytic reaction to occur. Suitable catalytic components include, but are not limited to, for example, catalysts, co-catalyst, ligands, substrates, aptamers, additives, acids, bases, H-donors, H-acceptors, etc. that are necessary for the catalytic reaction. In some embodiments, these catalysts include photocatalysts, transition metal catalysts, organic radical catalysts, organocatalysts, Lewis acid catalysts, Lewis base catalysts, among others. In some embodiments, ligands include, but are not limited to, metal binding ligands, pyridines, bipyridines, phosphines, and terpyridines. In some embodiments, substrates include, but are not limited to, amines, imidazoles, heterocycles, aryl bromides, boronic acids, bases, acids, carboxylic acids, ketones, aldehydes, and acid chlorides.

In one embodiment, there may be a molecular linker that tethers catalytic components to single stranded polynucleotides. In some embodiments, the first, second and at least third catalytic component are linked via a linker to their associated single stranded polynucleotide. The term linker therefore refers to any means by which the catalytic component can be tethered, covalently, or non-covalently linked to the polynucleotide. In some embodiments, this linker includes flexible linkers. The linkers can have variable lengths and flexibility and can also be varied in the library to provide additional flexibility in formation of the catalytic active site. Suitable flexible linkers are known in the art, and can include, for example, C5-C-30 alkyl or polyethylene glycol (PEG) chains. In other embodiments these linkers also include polysaccharide linkers, poly-lactic acid linkers, poly(lactic-co-glycolic acid) linkers, polypeptide linkers, or any linker commonly used in antibody-drug conjugates, including cleavable linkers. Suitable linkers are discussed in Bargh et al. “Cleavable linkers in antibody-drug conjugates,” Chem. Soc. Rev., 2019, 48, 4361-4374, incorporated by reference in its entirety regarding linkers.

In one preferred embodiment the linker molecule length may be from about 1 angstrom to about 100 angstroms, preferably about 1 angstrom to about 50 angstroms in length but may be longer or shorter depending on the application.

In another embodiment, the substrate may be tethered to the polynucleotide nanoscaffold and another substrate. For example, the substrate may further be linked or tethered to an additional substrate comprising a reporter molecule, or the substrate may be non-covalently associated with the additional substrate (e.g., free-floating). As described in more detail below, the ability to cleave the substrate and/or the additional substrate from the polynucleotide nanoscaffolds described herein allows for the ability to detect catalytic activity within the library during screening and testing.

To identify successful catalytic library combinations, a reporter molecule may be used. In one preferred embodiment the reporter molecule is biotin. The term reporter molecule refers to a molecule that can be detected by means known in the art, e.g., enzymatic activity, binding activity, fluorescent activity, etc. Suitable reporter molecules include, but are not limited to, for example, biotin, a fluorophore, quenchers/fluorophore pairs (e.g. FRET readout), luciferase, magnetic nanoparticle, among others. In other embodiments the reporter molecule may be a fluorophore. In still other embodiments the reporter molecules are quenchers or fluorophore pairs, such as those used in Forster resonance energy transfer (FRET). In another embodiment the reporter molecule is a magnetic nanoparticle that may allow for magnetic isolation. In one embodiment, the reporter molecule is added to a successful catalytic combination via a catalytic reaction. In another embodiment, the reporter molecule is removed from a successful catalytic combination by a catalytic reaction. In yet another embodiment, the reporter molecule could be added by way of binding to a change in functional group that allows for the reporter molecule to bind.

The reporter molecule may be conjugated to the polynucleotide nanoscaffold. A range of attachment chemistries may be used. Exemplary chemistry includes, when the reporter molecule is biotin, for example, condensation reactions with aldehyde- or ketone-bearing polynucleotides by alkoxyamine-biotin or hydrazide-biotin. Other conjugation chemistries include DMTMM and EDC couplings to attach carboxylic acid-bearing reaction components to amine-modified polynucleotides, as provided in Example 2. Other bioconjugation approaches may also be used, including Click chemistry.

The term “polynucleotide barcode” as used herein refers to a unique polynucleotide (e.g., DNA/RNA) sequence that is about 1-30 base pairs, preferably 2-30 (e.g., 2, 3, 4, 5, 6, 7, 8, 9, 10, 11, 12, 13, 14, 15, 16, etc.) that has a unique sequence that allows for identification of the catalytic component that is being tethered to the single stranded polynucleotide sequence containing the barcode. Just like the unique pattern of bars in a universal product code (UPC) identities each consumer product, a “DNA barcode” has a unique pattern of DNA nucleotides that can identify each catalytic component that is added to the polynucleotides making up the self-assembled polypeptide nanoscaffolds described herein. Suitable DNA barcodes are known in the art and can readily be designed by one skilled in the art. A barcode signature includes the indicative barcode of the catalytic active site of the self-assembled nanoscaffold. For example, a self-assembled nanoscaffold including three catalytic components in the catalytic active site each having an indicative barcode (including the first polynucleotide barcode, the second polynucleotide barcode, and the third polynucleotide barcode), the barcode signature would include three indicative barcodes.

As discussed above, the library comprises a plurality of polynucleotide nanoscaffolds. The term “polynucleotide nanoscaffold” or “polynucleotide structure” as used herein refers to the 3-D structure formed from the self-assembly of the single stranded polynucleotides (e.g., first, second, third etc. ss polynucleotides each comprising a catalytic component) to form a 3-D structure. The self-assembly ss polynucleotides that comprise the barcode and are linked to the catalytic component are also referred herein to as polynucleotide barcoded building block oligomers. Thus, the polynucleotide barcoded building block oligomers self-assemble by the binding of the complementary regions within each single strand to form a polynucleotide nanostructure assembly. The polynucleotide (DNA/RNA) nanostructures with diverse sizes and shapes, including G-quadruplexes, 3-way junctions, 4-way junctions, 5-way junctions, etc., and tetrahedra (e.g., as shown in the Examples and FIG. 6) are contemplated. The structural diversity of these DNA architectures and the precise tunability of the catalyst attachment sites enables a systematic investigation of which catalyst orientations are optimal for fast reactivity. The flexibility of the DNA scaffolds can be optimized by introducing base-pair mismatches, and the tethers or linkers connecting the abiotic catalysts to the DNA can be varied in length and flexibility. The reaction can further be accelerated through installation of a substrate binding site close to the at least two catalyst components, thus mimicking a common mechanism by which enzymes accelerate reactions.

In some embodiments, the self-assembling nucleic acid sequence forms at least three hairpin structures to provide the three-dimensional structure of the polynucleotide nano-structure scaffold. In a further embodiment, the self-assembling nucleic acid sequence forms at least four hairpin structures to provide the three-dimensional structure of the polynucleotide nano-structure scaffold. However, other shapes of structures are contemplated, including three-hairpin, five-hairpin, six-hairpin, etc. structures.

In the self-assembly of the polynucleotide nanoscaffolds, multiple catalytic components are brought together in sufficiently close proximity and orientation for a catalysis to occur. In one preferred embodiment the nanoscaffold brings at least 2, preferably at least 3 catalytic components in sufficiently close proximity and orientation for a catalysis to occur. Thus, in some embodiments, 2-50 catalytic components, alternatively 2-20 catalytic components can be brought into close proximity.

In one embodiment the catalytic components are brought into sufficiently close proximity within a catalytic active site in the three-dimensional polynucleotide nanoscaffold. In some embodiments, the catalytic active site may be a cavity within the three-dimensional structure formed by the assembling of the ss polynucleotides. In some embodiments, the catalytic active site is about 10 angstroms to about 200 angstroms. In some embodiments, the catalytic active site allows for the assembly of the catalytic components into close proximity with each other.

In other embodiments, the catalytic components are not enclosed in a 3-dimensional cavity within the 3-D polynucleotide scaffold. However, the catalytic components are still brought in sufficiently close proximity and orientation for a catalytic reaction to occur that is associated with the polynucleotide scaffold, for example, by a T-junction orientation of the polynucleotide nanoscaffold to bring the components tethered to the ss polynucleotides in close proximity. The catalytic system library can comprise a plurality of self-assembled polynucleotide nanoscaffolds. In terms of self-assembled polynucleotide nanoscaffolds, the term plurality comprises at least 10 or more unique self-assembled polynucleotide nanoscaffolds, at least 100 or more, preferably at least 1000 or more, more preferably at least 10,000 or more self-assembled polynucleotide nanoscaffolds. The ability to use the polynucleotide barcoding system allows for the ability to screen large numbers of combinations in one pot, for example, the library may contain at least 10⁵ self-assembled polynucleotide nanoscaffolds, at least 10⁶ self-assembled polynucleotide nanoscaffolds, at least 10⁷ self-assembled polynucleotide nanoscaffolds, at least 10⁸ self-assembled polynucleotide nanoscaffolds, or more within the single library. This allows for the ability to screen an enormous amount of different conditions in one-pot.

### Methods

A rapid method of assembling a catalytic system screening library and methods of using the library for screening are provided herein. This polynucleotide-based platform for combinatorial assembly and screening of millions of combinations of reactive abiotic functional groups provides the benefits of accelerating the discovery and development of catalytic reactions and supramolecular catalysts. The polynucleotides are used as both a structural unit to guide the combinatorial self-assembly of a large and diverse library of catalyst structures as well as barcoding tags to identify successful catalyst motifs in a post-screening sequencing readout. The platform is composed of two phases: combinatorial library assembly and library screening. A variety of screening approaches enable application of the library to different reaction types (bond-forming, bond-breaking, etc.)

Contemporary screening approaches focus on miniaturization and automation but remain fundamentally limited by diseconomies of scale. A combinatorial, self-assembling approach allows catalyst discovery to operate in an economy of scale where small increases in the number of individual components lead to exponential increases in the number of library members screened. Polynucleotides enable this both through sequence-dependent self-assembly and genetic barcoding allowing the labeling of the catalyst components within the structure.

The catalytic system screening library provides several advantages over prior methods, as the polynucleotide-based scaffolds can be used for pre-organization of abiotic functional groups into close proximity to accelerate reactions. The facile attachment of abiotic catalysts to DNA strands; barcoding to allow the characterization of synthetically-modified DNA; and self-assembly of the modified DNA strands to create millions of catalytic scaffolds allows for the ability to screen a plurality of scaffolds in a single reaction (greater than 10⁸ different scaffolds can be screened in one reaction).

In one embodiment, the present disclosure provides a rapid method of assembling a catalytic system screening library, the method comprising (a) obtaining a first, second and at least third single stranded polynucleotide oligomer, each single stranded polynucleotide oligomer comprising a portion complementary to each of the other first, second and at least third single stranded polynucleotide to drive self-assembly into a self-assembled polynucleotide nanoscaffold and distributing the first second and third single stranded polynucleotide oligomer each into a first, second and third plurality of containers; (b) adding to each container of the first, second or third plurality of containers a unique catalytic component capable of attaching to the single stranded polynucleotide, a unique polynucleotide barcode, and a polynucleotide ligase enzyme capable of ligating the polynucleotide barcode to the single stranded polynucleotide, to form a plurality of polynucleotide barcoded building block oligomers; (c) combining in a single reaction vessel the plurality polynucleotide barcoded building block oligomers from the first, second and at least third plurality of the containers to self-assemble into a plurality of polynucleotide nanoscaffolds to form a catalytic system screening library.

As described above, the polynucleotide barcoded building block oligomers self-assembly the binding of the complementary regions within each single strand to form a polynucleotide nanostructure assembly. The ligase present in the reaction mixture allows for the ligation of the unique polynucleotide barcode to each single stranded polynucleotide strand and is used to identify the catalytic component that is tethered to the single stranded (ss) polynucleotide prior to assembly of the nanoscaffolds containing multiple components. This self-assembly is performed under conditions favorable for the self-assembly of DNA nanostructures, including appropriate concentrations of metal ions and appropriate pH.

In some embodiments, a purification step is provided when making the polymeric building blocks (small molecules attached to ss polynucleotide strands). This purifications in some cases is to remove excess small molecule and unreacted DNA (with no small molecules attached).

In some embodiments, after we have assembled the libraries of polymeric nanoscaffolds, we could perform a purification to separate the fully assembled DNA scaffolds from any unreacted building blocks (which are individual strands of DNA).

In some embodiments, the polynucleotide barcode is attached to the 5′ end of the single stranded oligomer using a DNA ligase enzyme. In some embodiments, the single stranded polynucleotides (e.g., ss DNA oligomers) are synthesized with a position modified by a bioconjugation handle. The bioconjugation handle is the location at which the catalytic component may be tethered to the ss polynucleotide.

Step (c) comprises combining in a single reaction vessel the plurality polynucleotide barcoded building block oligomers from the first, second and at least third plurality of the containers to self-assemble into a plurality of unique polynucleotide nanoscaffolds to form a catalytic system screening library. Once the plurality of unique polynucleotide nanoscaffolds are self-assembled, the self-assembled ss polynucleotide strands can be covalently linked using a DNA ligase enzyme. In some embodiments, the method of assembling the library further comprises (d) ligating the plurality self-assembled polynucleotide nanoscaffolds in the catalytic system screening library in order to ligate assembled ss polynucleotides into a single polynucleotide structure that is capable of being sequenced, wherein the library comprising a plurality of self-assembled polynucleotide nano-scaffold each comprising a unique combination of catalytic components. While the library could work without this ligation step, during the screening method, the polymerase chain reaction (PCR) would not get one continuous read showing all the barcodes within each polynucleotide nanoscaffold allowing for identification of successful combinations of catalytic components. However, the method without ligation would still determine individual abiotic groups most abundant in the reaction and thus can be used to determine the best components for reaction.

One step of the creation of a polynucleotide nanoscaffold library may include an optional ligation of the polynucleotide nanoscaffold. In one embodiment each of the polynucleotide nanoscaffolds in the library is ligated so that downstream amplification and polynucleotide sequencing yield a continuous read and report the catalytically successful combinations of catalytic components. In another embodiment, the polynucleotide nanoscaffolds are not ligated and the downstream amplification and sequencing determine abundance of catalytic components, but not the particular combinations.

The reaction to self-assemble the first, second and at least third polynucleotide barcoded building block oligomers into the nanoscaffold requires a plurality of containers as each of the single stranded components are processed to add the catalytic component and the barcode in separate containers. Suitable containers for carrying out the reactions described herein are known in the art and include tubes, wells in multiwell plates (e.g., 24-well, 48-well, 96-well, 384-well, etc. plates), nanotubes and nanochannels and the like. The number of containers used with depend on the catalyst being tested and the number of catalytic components to be tethered to the ss polynucleotides. In some embodiments, 20 or more containers are used, alternatively 50 or more containers, alternatively 100 or more containers, alternatively 1000 or more containers depending on the number of ss polynucleotides being assembled and the number of catalytic components being screened. In some embodiments, 10-5000, 20-500, 20-200 containers are used, but the present invention is not limited in scope by the number of containers and include any number or ranges in between those contemplated.

Suitable polynucleotide ligase enzymes can ligate the polynucleotide barcode to the single stranded polynucleotide are known in the art and include, for example, DNA ligase, RNA ligase, and the like (e.g. T4 DNA ligase, T4 RNA ligase, etc.).

The single reaction vessel can be any suitable reaction vessel known in the art, including polymeric reaction vessels, glass reaction vessels and the like. Suitably, the reaction vessel is inert and does not interfere with the catalytic reaction occurring in the vessel.

As described above, in some embodiments, at least one of the catalytic components is a substrate, and wherein the substrate is attached to a reporter molecule, such as biotin. The ability to add a report molecule becomes important when using the library for screening, as discussed below, as the reporter molecule allows for the easy isolation of catalyst nanoscaffolds that are capable of catalytic activity where the barcodes allow for identification of the catalytic components within that nanoscaffold.

Thus, in some embodiments, the method further comprises (e) screening the library to identify polynucleotide nanoscaffolds with catalytic activity, thereby identifying the at least first, second and third catalytic component that have catalytic activity with the catalyst.

The term screening as used herein refers to the (a) testing or exposing the library under conditions that would facilitate a catalytic reaction and (b) identifying the catalytic components within the polynucleotide nanostructures that were capable of catalytic activity.

The testing or exposing step is easily carried out by one skilled in the art under conditions that would allow for catalytic activity. Preferably, the testing is carried out such that a reporter molecule is able to be detected or a reporter molecule allows for the isolation and separation of the polynucleotide nanoscaffolds that demonstrate catalytic activity from those that did not show catalytic activity. Some examples of these testing methods are described in the examples below. However, the present invention is not limited by these examples and it is contemplated that a number of different testing method and reporter molecules and methods can be used in order to test and isolate the polynucleotide nanoscaffolds that demonstrate positive catalytic activity.

In some embodiments, the catalyst system library made by the method described herein is provided.

In further embodiments, a high-throughput method of screening a catalyst system library, wherein the method comprises: (a) exposing the library of described herein under catalytic reaction conditions to obtain a reaction; and (b) detecting the self-assembled polynucleotide nanoscaffolds that undergo a catalytic reaction in step (a) to identify catalytic components that have activity in combination.

In some embodiments, step (a) comprises incubating the single reaction vessel under conditions that allow for catalytic activity. These conditions may differ depending on the catalytic components and can be determined by one skilled in the art. In some embodiments, different catalytic conditions (e.g., temperature, pH, etc.) can be used with the same catalytic library to further determine the best reaction conditions. In some embodiments, different metals can be tested with the same catalytic library, in which the library members consist of different metal-binding functional groups.

The catalytic components detected can be evaluated on whether they catalyze the reaction efficiently in the absence of DNA scaffolding. If the components serve as an efficient catalytic system without the DNA nanoscaffold, this is advantageous: the DNA nanoscaffold will have enabled discovery of DNA-free catalytic reactions that operate under mild conditions. If the DNA nanoscaffold is required for efficient catalysis, this is also advantageous: it will indicate that the DNA nanoscaffold mimics enzymes by accelerating reactions through supramolecular pre-organization. Both are contemplated in this invention.

In one embodiment, step (b) comprises: (i) isolating polynucleotide nanoscaffolds with successful catalytic activity; and (ii) optionally amplifying the polynucleotide nano-scaffolds of (i); and (iii) sequence the polynucleotide to determine the polynucleotide barcodes contained within each polynucleotide nano-scaffold with catalytic activity, wherein each polynucleotide barcode identifies each of the catalytic components within the polynucleotide nano-scaffold. In some embodiments, step (iii) is next generation polynucleotide sequencing.

In some embodiments, step (i) comprises: (a) isolating polynucleotide nanoscaffolds comprising a reporter molecule, wherein attachment of the reporter molecule is associated with a catalytic activity. There are multiple ways contemplated in which the reporter molecule is used in the method of identifying the positive catalytic activity. In one embodiment, the reporter molecule could be added via the catalytic reaction. In another embodiment, the reporter molecule is added by way of binding to a change in a functional group that allows for the reporter molecule to bind to the polynucleotide scaffolds that had catalytic activity.

For example, but not limited to, in cases in which biotin is used as a reporter molecule, the biotin can be 1) added to the polynucleotide nanoscaffold during the catalytic reaction. In this scenario, the polynucleotide nanoscaffolds with positive catalytic activity can then be isolated via binding to streptavidin (e.g., streptavidin column, plate, etc.) and the isolated polynucleotide scaffolds can undergo next generation sequencing.

In some embodiments, PCR is carried out to amplify the positive polynucleotide scaffolds before next generation sequencing is performed. In a further example, 2) the reporter molecule, e.g., biotin, may be a catalytic component or may be covalently linked to a catalytic component. In this example, when the catalytic activity occurs, the biotin can be cleaved from the polynucleotide scaffold, and the positive polynucleotide scaffolds again can be isolated from the un-reacted scaffold by streptavidin, this time isolating those nanoscaffolds that do not bind to streptavidin.

Thus, in some examples, the biotin-labeled polynucleotide scaffolds will be isolated from the mixture using beads coated with the protein streptavidin, which binds very tightly to biotin. The DNA nanostructures captured on the beads will be amplified by polymerase chain reaction (PCR), using a procedure that is tolerant to abiotic groups on the DNA. The resulting amplified double-stranded DNA will be analyzed using next-generation sequencing, a highly quantitative method that allows millions of DNA sequences to be detected.

In some further embodiments, step (i) comprises: (a) isolating polynucleotide nano-scaffolds lacking a reporter molecule, wherein detachment of the reporter molecule from the scaffold indicates catalytic activity, and wherein the catalytic library comprising polynucleotide-nanoscaffolds each comprising a reporter molecule attached prior to exposure to catalytic reaction conditions.

In some embodiments, step (ii) comprising PCR amplification of the polynucleotide sequences prior to sequencing. PCR can use primers designed to amplify the polynucleotide scaffolds and allows for an increase detection of positive scaffolds that may have catalytic activity but may not be as robust as others.

In some embodiments, the step of isolating is via affinity purification. In other embodiments, the isolating comprises exposing the reacted library to a streptavidin purification column to isolate the polynucleotide nano-scaffolds either having biotin bound or not having biotin bound.

The methods described herein can be used iteratively, e.g., the positive catalytic polynucleotide scaffolds isolated from one combinatorial library of catalytic structures can undergo the process again using additional catalytic components to generate an additional library displaying different characteristics than the first. Thus, this process is iterative, the new catalysts discovered can be progressively improved in their activity by creating new libraries that contain updated abiotic groups (considering what is learned from the first screening).

In some embodiments, one step of the use of the polynucleotide nanoscaffold library includes the use of a reporter molecule. In one preferred embodiment, the reporter molecule is added to a successful catalytic combination via a catalytic reaction. In another embodiment, the reporter molecule is removed from a successful catalytic combination by a catalytic reaction. In yet another embodiment, the reporter molecule could be added by way of binding to a change in functional group that allows for the reporter molecule to bind.

High-throughput catalytic screening using combinatorial libraries of polynucleotide nanoscaffolds has the potential to screen many types of catalytic reactions. In three embodiments these catalytic reactions include copper-TEMPO (TEMPO=2,2,6,6-tetramethylpiperidine-N-oxyl) alcohol oxidation, bimetallic catalysis, palladium catalysis, debenzylation catalysis, nickel-photoredox catalysis, and discovery of catalysts for the degradation of chemical warfare agents.

High-throughput catalytic screening using combinatorial libraries has many potential applications. In one embodiment the successful catalytic combinations can be used to build, train, and test a computer model that may be able to predict successful catalytic combinations that are not present in the original combinatorial library. In one preferred embodiment the computer model is a machine learning model.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The invention will be more fully understood upon consideration of the following non-limiting examples.

### EXAMPLES

### Example 1: Polynucleotide Nanoscaffold Catalysts: Combinatorial Platform to Screen 10⁸ Catalysts

This Example demonstrates the design of supramolecular synthetic catalysts having architecture in which multiple reactive groups are displayed on a 3D scaffold. In this example, DNA is used as a building material for the creation of supramolecular enzyme mimicking catalysts providing predictable self-assembly of 3D nanostructures featuring cavities with diverse sizes and shapes, diverse abiotic groups can be attached site-specifically on supramolecular DNA architectures, and DNA encodes information and thus enables high-throughput combinatorial reaction discovery.

Conventional catalyst discovery involves low throughput testing of substrates, catalysts, co-catalysts, and additives. This example demonstrates a DNA nanoscaffold platform in which 10⁸ supramolecular catalysts, each displaying distinct abiotic groups can be synthesized in a single test tube. Using tools from biotechnology, we will rapidly identify the most active catalysts from the mixture.

The information-encoding properties of DNA make it extremely useful as a platform for high-throughput screening and chemical discovery. DNA-mediated or -encoded discovery has been used for a variety of functions in non-natural contexts. DNA oligonucleotides have been evolved for the discovery of selective small-molecule binders. DNA templates have been exploited to create highly functionalized DNA polymers' and to control preparation of synthetic polymers. Most famously, DNA-encoded libraries have been developed for discovery of pharmaceutically active small molecules.

Additionally, “DNAzymes” have been discovered through high-throughput screening, including ones that incorporate abiotic components for catalysis. While relatively few DNAzymes have been evolved for synthetic applications, evolution of DNA sequences has been demonstrated to tune Copper (Cu) redox potential, as observed in Dipankar Sen's Cu-dependent DNAzyme for Cu-catalyzed azide-alkyne cycloadditions in the absence of added reductants.

DNA has not yet been pursued as a platform for supramolecular catalyst discovery in which >2 synergistic catalytic groups are held in proximity. This application demonstrates how to make and use an unprecedented application of DNA as an enzyme-mimicking scaffold to accelerate abiotic reactions, while also enabling combinatorial screening due to the information-encoding properties.

### Combinatorial Platform to Rapidly Screen 10⁸ Catalysts Under Mild Conditions

The discovery of new catalytic transformations that operate under mild conditions is important for advancing energetic materials synthesis. Efficient and scalable debenzylation reactions, for example, would streamline the synthesis of energetic precursors. Reactions involving C—N bond formation are also of interest for the synthesis of cluster-like energetic molecules. We developed a combinatorial platform to discover mechanistically diverse catalytic reactions that operate under mild conditions. In Example 1.1, we test our combinatorial platform using Cu-TEMPO alcohol oxidation as a proof-of-principle reaction. In Example 1.2, we will extend the platform to the discovery of bond-breaking reactions—specifically, debenzylation of amines under mild conditions. In Example 1.3, we will apply the platform to discover transition metal-photoredox C—N bond forming catalysts, in reactions where amides or amines serve as the nucleophiles.

### Example 1.1. Develop and Validate the Platform using Cu-Nitroxyl Alcohol Oxidation

A library of 10⁸ DNA nanoscaffolds will be synthesized in a single test tube, with each library member bearing distinct abiotic functional groups and unique DNA barcodes (FIG. 6). To validate the platform, we will initially focus on copper-catalyzed alcohol oxidation as the target reaction, and thus each DNA nano-scaffold will display 4 distinct abiotic catalytic component groups in the central cavity: a copper catalyst, an organic radical catalyst, a nitrogen-containing base (known to accelerate this reaction), and an alcohol substrate. Copper-mediated alcohol oxidation is a promising proof-of-principle reaction because the library will consist of “positive control” nanoscaffolds expected to exhibit the desired reactivity, along with nanoscaffolds bearing alcohols that are difficult to oxidize. In a single experiment, our workflow of combinatorial synthesis followed by high-throughput testing will yield detailed structure-property relationships describing which catalytic components are optimal for oxidation of different alcohol substrates.

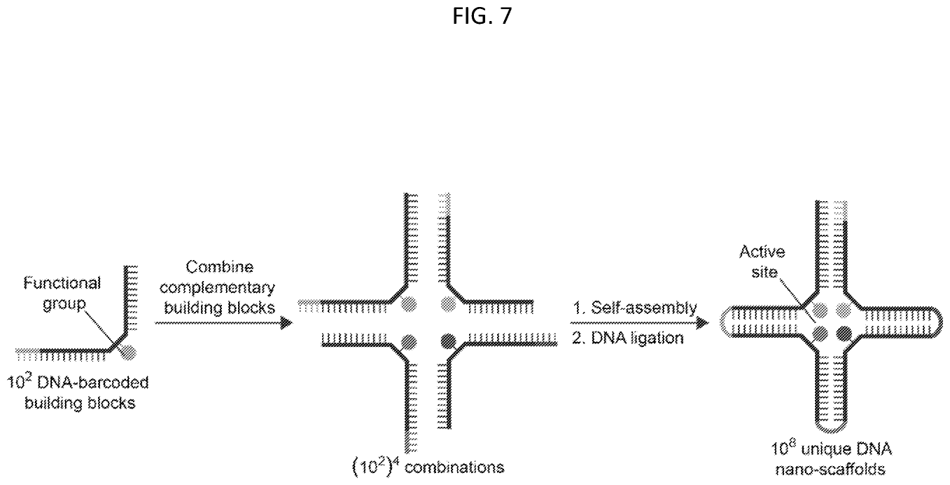

FIG. 7 illustrates how a library of 10⁸ candidate DNA-based catalysts, bearing unique DNA barcodes, will be assembled in a single test tube. Four different single-stranded (ss) DNA oligomers will be synthesized, with a central position modified by a bioconjugation handle (e.g., a place at which the catalytic component is added). The four ssDNA oligomers will possess appropriate sequence complementarity to drive assembly of a DNA 4-way junction. Next, each DNA oligomer will be split into 100 different tubes. In each tube, a distinct functional group will be attached to the bioconjugation handle, and a unique DNA barcode will be simultaneously attached to the 5′ end of the DNA oligomer using a DNA ligase enzyme. All 4×100 unique oligomers will be mixed in a single tube, and DNA hybridization will drive the self-assembly of (10²)⁴=10⁸ unique DNA nano-scaffolds. After self-assembly, the four ssDNA strands within each nano-scaffold will be covalently linked using a DNA ligase.

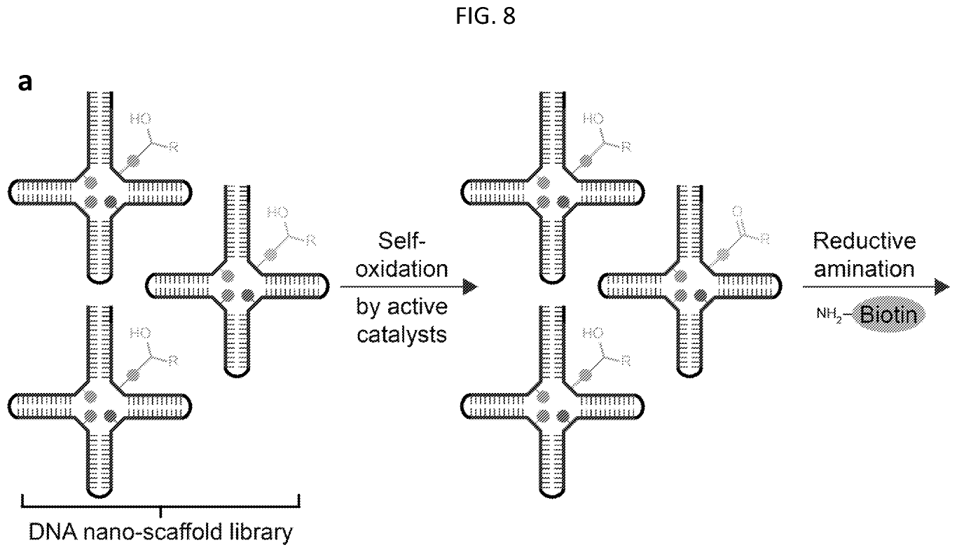

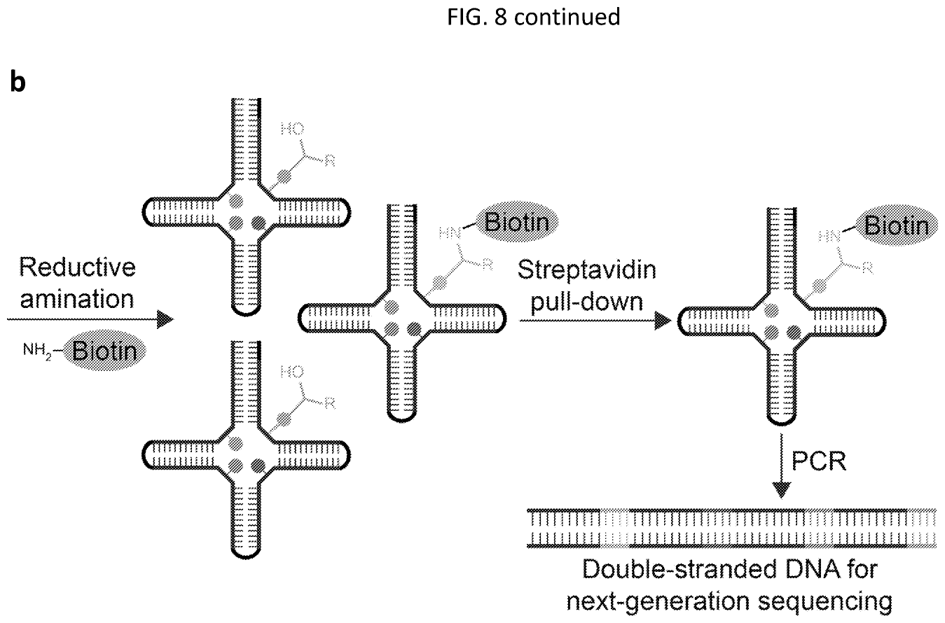

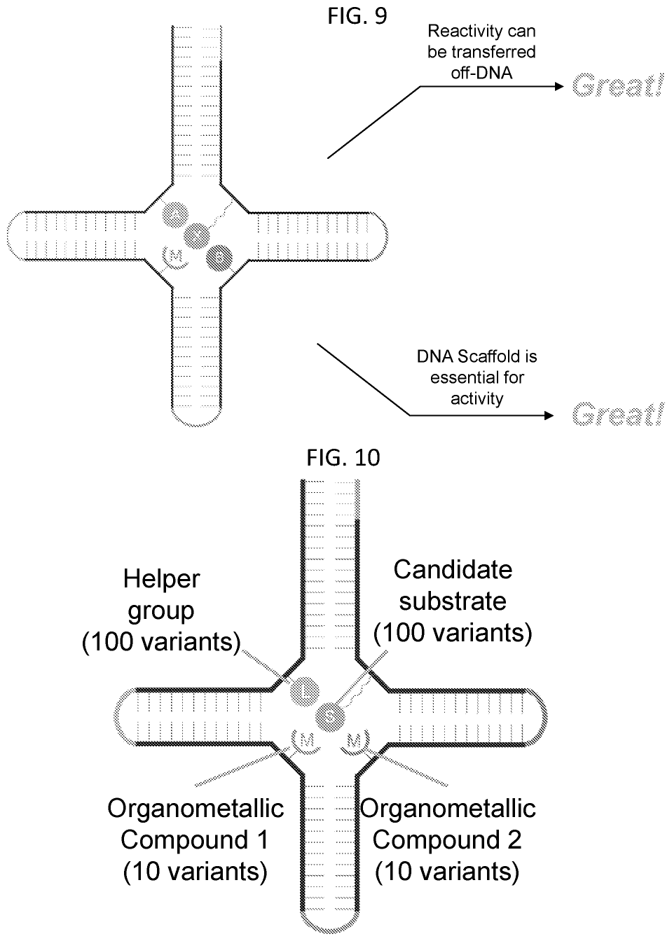

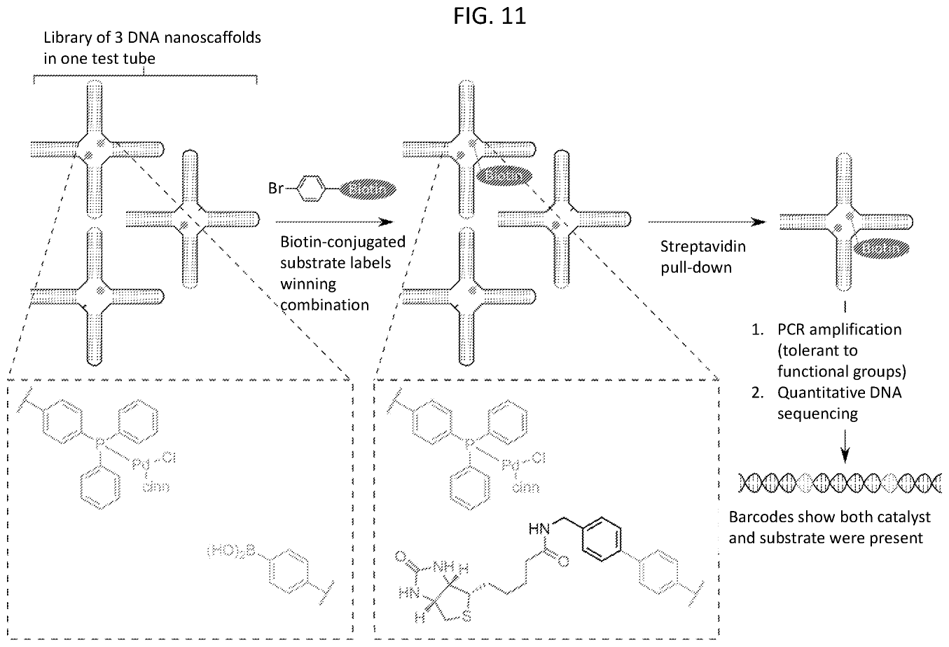

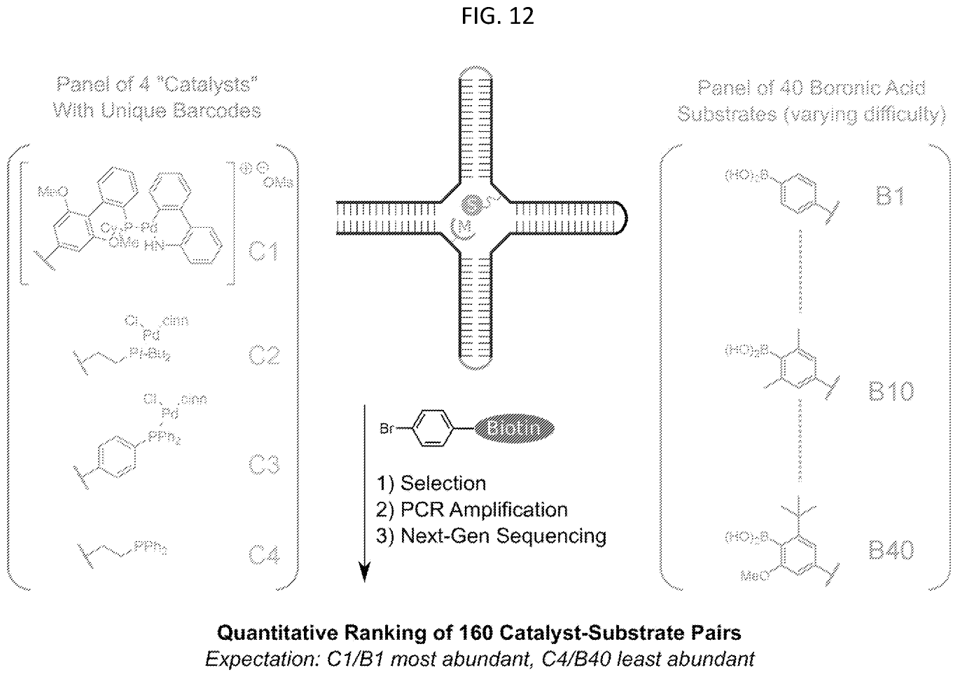

A high-throughput selection strategy to rapidly isolate the DNA nano-scaffolds exhibiting catalytic activity will be implemented. As depicted in FIG. 8a and FIG. 8b, DNA nano-scaffolds with high activity will oxidize the appended alcohol. Next, a small molecule consisting of an amine attached to biotin will be added to the reaction mixture. Through reductive amination, biotin will be attached to scaffolds bearing aldehydes or ketones, but not scaffolds bearing the unreacted alcohol. The biotin-labeled DNA scaffolds will be isolated from the mixture through affinity purification; the winning DNA nanostructures will be amplified using PCR (following a procedure tolerant to abiotic groups on the DNA); and the resulting amplified double-stranded DNA will be analyzed using next-generation sequencing.

The DNA sequencing results will be decoded to determine which DNA nanoscaffolds were enriched during the selection. The abiotic groups on each nanoscaffold are read based on the DNA barcodes. Given the size and complexity of the catalyst library, there may be hundreds or even thousands of winners, and structural trends may not be discernable upon manual inspection. Molecular descriptors, such as polarity and size, will therefore be assigned to each abiotic group to aid in discerning trends. Promising catalyst structures will be synthesized on a larger scale and characterize them for catalytic efficiency and longevity.

Importantly, the ability of promising catalysts to oxidize a free-floating alcohol substrate (not tethered) will be evaluated. The DNA sequences near the central cavity (e.g., catalytic activity site) will be systematically varied to alter its size and flexibility, then the effect on catalytic activity will be studied. Mechanistic insights from these experiments will inform the design of second-generation nanoscaffold libraries, and the selection process will be repeated. This iterative method will allow further refinement via machine learning (ML), thus mimicking natural evolution.

The abiotic reactants will be evaluated on whether they catalyze the reaction efficiently in the absence of DNA scaffolding. If the abiotic components serve as an efficient catalytic system without the DNA nanoscaffold, this is advantageous: the DNA nanoscaffold will have enabled discovery of DNA-free catalytic reactions that operate under mild conditions. If the DNA nanoscaffold is required for efficient catalysis, this is also advantageous: it will indicate that the DNA nanoscaffold mimics enzymes by accelerating reactions through supramolecular pre-organization. In this scenario, we will carefully study the mechanisms of the enzyme-mimicking catalysts and fine-tune their structures to achieve even greater rate accelerations. Additionally, these enzyme-mimicking catalysts will be optimized to achieve stereo- and regio-selective reactions.

Numerous steps of the workflow have been validated, including DNA barcoding, combinatorial self-assembly of modified DNA nanostructures, and enzymatic ligation to join individual DNA strands in the self-assembled nanostructures. Furthermore, the key step has been achieved: DNA scaffolds bearing a copper catalyst can oxidize a self-appended alcohol to an aldehyde, which we have subsequently attached to biotin via reductive amination. Inter-scaffold reactivity can be avoided by performing selections under dilute conditions (below 60 nM). We have also optimized conditions for pull-down of biotinylated DNA nanoscaffolds and PCR amplification of chemically-modified DNA. Finally, we have developed protocols for next-generation DNA sequencing and quantitative analysis of the barcodes.

The present platform allows for multiple alternative DNA scaffolds, synthetic tethers, DNA polymerases, and catalyst selection strategies. As a backup reaction that is mechanistically related, we will expand the approach toward C—N bond-forming methodology. We will expose the Cu-nitroxyl DNA nanoscaffold library directly to the biotin-amine probe to evaluate which catalysts are capable of oxidative alcohol-amine coupling (to form amides).

### Example 1.2. Catalysts for Debenzylation of Protected Amines

The selective removal of benzyl protecting groups from amines under mild conditions remains an outstanding challenge in organic synthesis. Commonly used reductive hydrogenolysis conditions necessitate the use of precious, noble metals and the harsh conditions limit the functional group tolerance of the method. A further challenge is introduced when the method necessitates the removal of just one benzyl group from a dibenzylated amine, or the selective removal of one benzyl group from a substrate containing multiple benzyl protecting groups. Oxidative methods for the debenzylation of amines represent a promising alternative to hydrogenolysis, and careful choice of oxidant can lead to some chemoselectivity. Methodologically diverse, these oxidative methods often rely on the use of stoichiometric oxidants such as DDQ, CAN, or hypervalent iodine reagents, and are only sometimes catalytically accelerated, making selectivity through catalyst-control challenging, instead relying on native substrate reactivity or subtle tweaking of reaction conditions to govern selectivity. Oxidative debenzylation using CAN has been demonstrated for complex cage-like amines such as a hexaazaisowurtzitane structure, suggesting that novel milder, catalyst-controlled oxidative debenzylation methods will be promising for complex polyamine structures.

Our platform can be used to identify a catalytic system that uses molecular oxygen as the terminal oxidant for the straightforward and selective debenzylation of amines. The commonly proposed mechanism for oxidative debenzylation is single-electron oxidation of the benzylamine followed by formal loss of an electron and a proton to generate the imine, which then hydrolyzes to give the unprotected amine and an aldehyde byproduct. Existing transition metal-mediated methodology for the oxidation of amines to imines using oxygen as the terminal oxidant is frequently challenged by harsh reaction conditions (above 100° C.) and reaction conditions that are not water compatible. In addition to ligand design to optimize reactivity, a common strategy is the use of co-catalytic systems.

Many of these methods incorporate additives and co-catalysts, the mechanisms are often complex, with poorly defined coordination equilibria, and they usually require heating above 100° C. For example, Bäckvall combined ruthenium catalysts with quinone and cobalt co-catalysts to oxidize amines to imines, but even though their conditions allowed for air to serve as the terminal oxidant, refluxing in toluene was still necessary. Similar approaches in Cu-catalyzed oxidations have yielded remarkable improvements in the mildness of reaction conditions. Cu-catalyzed amine oxidations often require high temperatures, but through choice of coordinating groups and nitroxyl radical co-catalysts Hu, Kerton, and Xu were able to demonstrate the preparation of imines under remarkably mild conditions using molecular oxygen as the terminal oxidant. Interestingly, no hydrolysis of the resulting benzylamines was observed, even in semiaqueous media. This suggests that the application of these oxidative methods towards debenzylation will require the development of novel reactive systems.

The myriad of catalytic systems (different metal centers, accelerating ligands, and additives) and the wide variety of substrate classes (dibenzylamines, cyclic benzylamines, primary and secondary benzylamines, aliphatic, activated, aromatic) make oxidative debenzylation a promising reaction for our DNA-scaffolded catalyst discovery platform, which will allow us to rapidly screen a wide variety of ligands, co-catalysts, and additives. The stringency of the reaction conditions will help us to select for DNA-scaffolded catalysts that enable mild, aqueous, DNA-compatible transition metal catalyzed oxidative debenzylation of amines using air as the terminal oxidant.

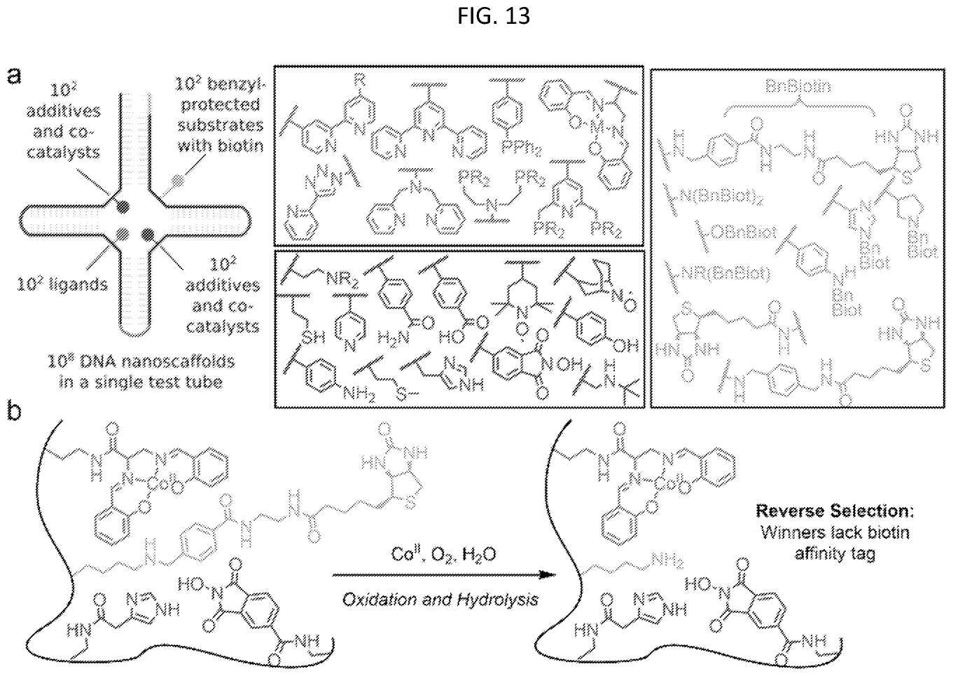

To develop this reaction, we propose to follow a similar workflow to that described in Example 1.1. A DNA library will be constructed (FIG. 13) that features a variety of metal-binding ligand architectures known to enable the oxidation of amines, a wide panel of co-catalytic groups known to promote the transition-metal-catalyzed oxidation of amines and alcohols, weak acids and bases to help facilitate proton transfer, and monodentate and labile coordinating groups to tune the coordination sphere of the catalytic metal. By assembling a combinatorial library with 100 ligands, 100 substrates, and 100 different additives at the two remaining positions of the DNA architecture, 10⁸ unique reaction combinations can be assayed for debenzylation activity under aqueous conditions. By screening a variety of substrate classes (di- vs. monobenzylated, primary vs. secondary, cyclic vs. acyclic, benzyl esters vs. benzyl amines, and a PMB-analogous substrate), we will gather data about the selectivity of each catalytic system and confirm generality. Ligands to be attached will include salens, phosphines, polypyridyl ligands, pincer ligands, and triazole-containing ligands. Additives and co-catalysts will include N-hydroxyimides, nitroxyl radicals, phenols, aromatic bases and secondary/tertiary amines (as both additives, and precursors to in situ N-oxidation), monodentate ligands, and a variety of acids and bases for proton transfer. Attachment to DNA will be performed using standard bioconjugation techniques (peptide couplings, CuAAC click chemistry, DNA-compatible reductive amination, and SuFEx click chemistry). In screening the library, multiple selections will be set up in parallel using different metal pre-catalysts (including Co, Cu, and Ru salts) that have been used for aerobic oxidations of amines under mild conditions at concentrations known to preserve the DNA's PCR amplifiability.

In contrast to the selection approach in Example 1.1, we propose to use a two-step selection. First, we will pursue a reverse selection where successful catalyst combinations will cleave a benzyl protecting group bearing a biotin affinity tag (FIG. 13). Upon exposure to streptavidin-beads, unsuccessful reaction combinations will bind, and the winning reaction combinations in solution can be isolated, amplified, and sequenced to identify the barcodes of the enabling catalyst components. At this point, a new, focused library will be prepared, and a second, positive selection will be implemented: we will synthesize a single substrate, a short perbenzylated polyhistidine tag, using standard solid phase peptide synthesis methods and attach this to the quadrant of the DNA nanoscaffold corresponding to the substrate. Successful catalysts during selection will cleave multiple benzyl groups, leading to increased affinity for Ni(NTA), allowing for both a positive, affinity-based selection as well as a screen for catalytic turnover. Successful catalysts will be reassembled and validated for the desired mild debenzylation activity.

We have already implemented a protocol for DNA-compatible reductive aminations (necessary for the attachment of some catalysts and preparation of some of the benzylated substrates), and we have confirmed the DNA compatibility of similar oxidative reaction conditions and performed reproducible alcohol oxidations. We have also optimized a protocol for the attachment of nitroxyl radicals to DNA on the library scale. Our already-developed methods for nanoscaffold assembly, ligation, amplification, and sequencing (vide infra) will be applied.

If debenzylation is too fast and the selection leads to too broad of a pull-down, we can reduce the availability of oxygen and the reaction time, and cool the reaction to increase the stringency. If air does not provide rapid enough oxidation, we can increase the pressure of oxygen or pivot to DNA-compatible stoichiometric oxidants such as CAN and hypervalent iodine reagents. While many stoichiometric oxidants are capable of catalyst-free debenzylation of amines, the stringency of the selection can be increased to exclude non-catalytic debenzylations. If the benzyl-biotin protecting group with affinity tag is too difficult to remove due to its decreased electron density on the ring, we can switch to a histamine aptamer positive selection where each scaffold is attached to benzylated histamine, and successfully debenzylated histamine-bearing scaffolds are isolated through binding to a histamine aptamer. Should the selection prove unsuccessful in identifying a combination of co-catalysts and additives that enable mild, aqueous debenzylations of amines, we can pivot to a library that includes photocatalysts and potential HAT additives known to facilitate debenzylations of ethers and amines.

### Example 1.3. Nickel-Photoredox Catalysts for C—N Bond-Forming Reactions

Nickel-photoredox catalysis is a broad and expanding area in synthetic methodology that enables a variety of reactions, including C—N bond-forming reactions. The mechanism of nickel-photoredox catalysis is generally proposed to proceed through rapid oxidative addition of an aryl halide to Ni⁰, followed by interception of a photocatalytically-generated alkyl radical by the Ni^{II}(Ar)(X) oxidative adduct. The resulting alkyl-aryl Ni^{III} undergoes reductive elimination, releasing product. The resulting Ni^{I} complex is then reduced by the photocatalyst to regenerate Ni⁰.

Nickel-photoredox catalysis is a promising reaction class to explore within our combinatorial platform because of the breadth of accessible reactions, and also because Molander recently demonstrated that nickel-photoredox can be performed under DNA-compatible, open-air conditions for C(sp²)-(sp³) cross-couplings. We will prepare a combinatorial DNA nanoscaffold library suitable for discovering DNA-compatible versions of known nickel-photoredox transformations, as well as for discovering novel reactivity.

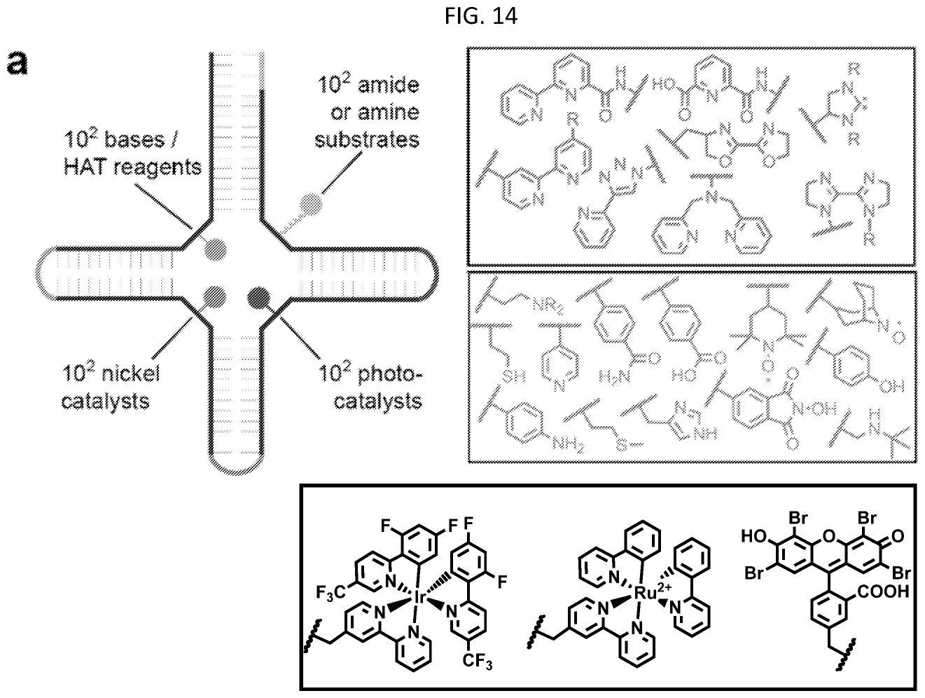

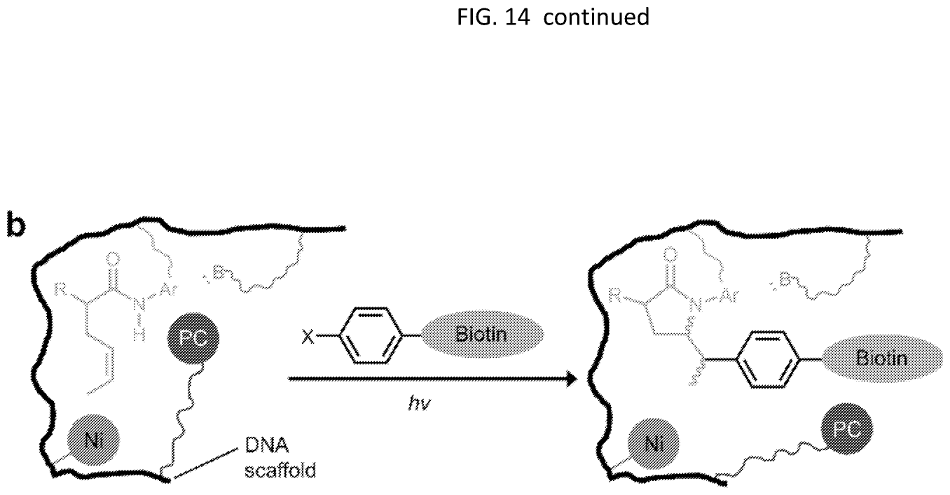

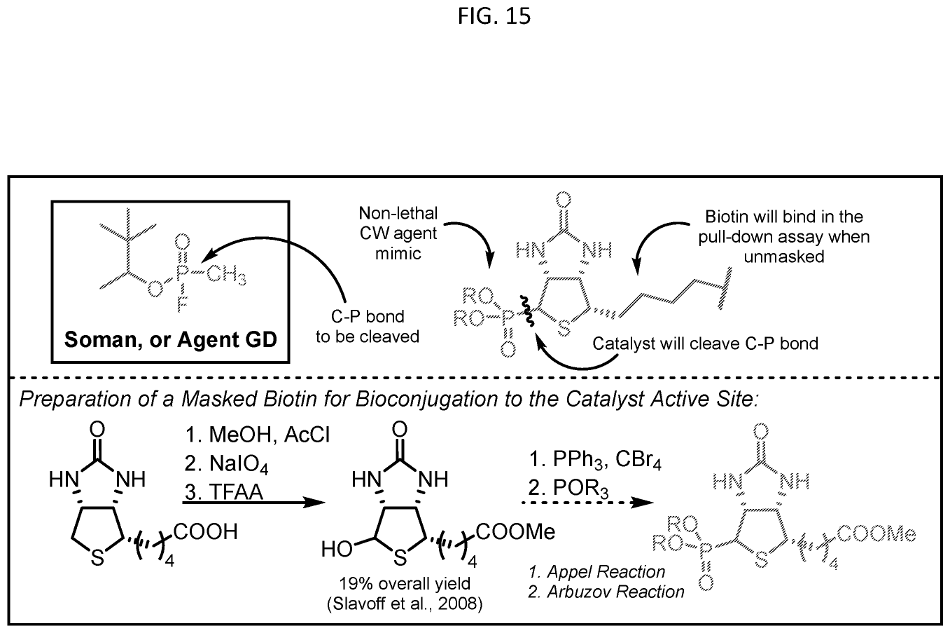

FIG. 14a and FIG. 14b depicts the composition of the nickel-photoredox DNA nanoscaffold library that we will prepare. The workflow for assembling this library will be the same as in Example 1.1. One DNA scaffold arm will be functionalized with diverse nickel ligands known to be active in cross-couplings. Another arm will be functionalized with photocatalysts, including Ir(dF(CF₃)ppy)₂bpy-type photocatalysts (ppy=2-phenylpyridine) that are typically used in Ni-photoredox reactions. Importantly, bioconjugation-compatible versions of these photocatalysts have been reported. A third arm will be appended to bases or hydrogen-atom transfer (HAT) reagents to facilitate proton-coupled electron transfer (PCET). Abiotic groups will be appended using the standard methodologies described above.

The identity of substrates appended to the fourth arm will depend on the specific reaction target. An example reaction for which we will pursue Ni-photoredox catalyst discovery is cascade amidoarylation of unactivated olefins (FIG. 14). This reactivity has been demonstrated in a conventional format by Molander using synergistic nickel-photoredox catalysts, in a cyclization of an amide reacting with an alkene, forming a radical that is subsequently coupled to an aryl halide. We will attach a panel of amide substrates bearing alkenes for amidoarylation. We synthesize the amide substrates starting from carboxylic acid precursors, then bioconjugate them to DNA through the aryl group on the amide nitrogen. Once the amide substrate-bearing library has been assembled, we will introduce an aryl halide attached to biotin; DNA nanoscaffolds exhibiting the desired reactivity will tag themselves with biotin, enabling pull-down, PCR, and DNA sequencing (as in Example 1.1).

As in Example 1.1, any winning DNA scaffolds identified through sequencing will be re-synthesized on larger scale. We will characterize their catalytic properties, including testing of whether they operate on a free-floating substrate for multiple turnovers, and whether catalytic rate enhancement is dependent on the DNA scaffold. All high-efficiency, DNA-compatible catalytic systems will be explored. Specifically, high-activity amidoarylation catalysts will be used directly for the functionalization of DNA nanostructures with chiral cavities to enable stereoselective cyclizations.

Synthesis of a carboxylate-bearing bipyridine ligand is currently in progress in our lab following the literature procedure by Pan et al. We have already synthesized the iridium precursor needed for installation of an attachment handle. We have successfully attached multiple photocatalysts (including Eosin Y and Ru(bpy)₃²⁺ analogs) using a variety of attachment strategies including isothiocyanate chemistry, Cu-click, and amide bond formation. We have already validated the photocatalytic activity of these DNA-conjugated photocatalysts for amine oxidation, azide reductions, and controlled polymerizations.

An admitted risk is that radical intermediates formed during catalysis would react detrimentally with DNA, quenching the desired reaction pathway. To mitigate this risk, we will perform catalyst selections under conditions previously reported to be DNA-compatible. If needed, we will add radical quenchers, which will decrease yield, but also minimize DNA damage. As an alternative, we can couple carbamates to unactivated secondary alkyl halides, as reported using a copper photoredox catalyst. We will also pursue the hydroamination of alkenes, which involves photocatalysis but not nickel catalysis.

### REFERENCES

1. Brenner, S. & Lerner, R. A. Encoded combinatorial chemistry. PNAS 89, 5381-5383 (1992).
2. Nielsen, J., Brenner, S. & Janda, K. D. Synthetic methods for the implementation of encoded combinatorial chemistry. J. Am. Chem. Soc. 115, 9812-9813 (1993).
3. Ellington, A. D. & Szostak, J. W. Selection in vitro of single-stranded DNA molecules that fold into specific ligand-binding structures. Nature 355, 850-852 (1992).
4. Yu, H., Alkhamis, O., Canoura, J., Liu, Y. & Xiao, Y. Advances and Challenges in Small-Molecule DNA Aptamer Isolation, Characterization, and Sensor Development. Angewandte Chemie International Edition 60, 16800-16823 (2021).
5. Ruscito, A. & DeRosa, M. C. Small-Molecule Binding Aptamers: Selection Strategies, Characterization, and Applications. Front Chem 4, 14 (2016).
6. Hili, R., Niu, J. & Liu, D. R. DNA Ligase-Mediated Translation of DNA Into Densely Functionalized Nucleic Acid Polymers. J. Am. Chem. Soc. 135, 98-101 (2013).
7. Snyder, T. M. & Liu, D. R. Ordered Multistep Synthesis in a Single Solution Directed by DNA Templates. Angewandte Chemie International Edition 44, 7379-7382 (2005).
8. McKee, M. L. et al. Programmable One-Pot Multistep Organic Synthesis Using DNA Junctions. J. Am. Chem. Soc. 134, 1446-1449 (2012).
9. Niu, J., Hili, R. & Liu, D. R. Enzyme-free translation of DNA into sequence-defined synthetic polymers structurally unrelated to nucleic acids. Nature Chem 5, 282-292 (2013).
10. Goodnow Jr, R. A., Dumelin, C. E. & Keefe, A. D. DNA-encoded chemistry: enabling the deeper sampling of chemical space. Nat Rev Drug Discov 16, 131-147 (2017).
11. Favalli, N., Bassi, G., Scheuermann, J. & Neri, D. DNA-Encoded Chemical Libraries: Achievements and Remaining Challenges. FEBS Lett 592, 2168-2180 (2018).
12. Neri, D. & Lerner, R. A. DNA-Encoded Chemical Libraries: A Selection System Based On Endowing Organic Compounds With Amplifiable Information. Annu Rev Biochem 87, 479-502 (2018).
13. Kunig, V., Potowski, M., Gohla, A. & Brunschweiger, A. DNA-encoded libraries—an efficient small molecule discovery technology for the biomedical sciences. Biological Chemistry 399, 691-710 (2018).
14. Flood, D. T., Kingston, C., Vantourout, J. C., Dawson, P. E. & Baran, P. S. DNA Encoded Libraries: A Visitor's Guide. Israel Journal of Chemistry 60, 268-280 (2020).
15. Yuen, L. H. & Franzini, R. M. Achievements, Challenges, and Opportunities in DNA-Encoded Library Research: An Academic Point of View. ChemBioChem 18, 829-836 (2017).
16. Zhao, G., Huang, Y., Zhou, Y., Li, Y. & Li, X. Future challenges with DNA-encoded chemical libraries in the drug discovery domain. Expert Opinion on Drug Discovery 14, 735-753 (2019).
17. Silverman, S. K. In vitro selection, characterization, and application of deoxyribozymes that cleave RNA. Nucleic Acids Research 33, 6151-6163 (2005).
18. Chandra, M., Sachdeva, A. & Silverman, S. K. DNA-catalyzed sequence-specific hydrolysis of DNA. Nat Chem Biol 5, 718-720 (2009).
19. Wang, M. et al. In vitro selection of DNA-cleaving deoxyribozyme with site-specific thymidine excision activity. Nucleic Acids Res 42, 9262-9269 (2014).
20. Sheppard, T. L., Ordoukhanian, P. & Joyce, G. F. A DNA enzyme with N-glycosylase activity. Proc Natl Acad Sci USA 97, 7802-7807 (2000).
21. Mohan, U., Burai, R. & McNaughton, B. R. In vitro evolution of a Friedel-Crafts deoxyribozyme. Org. Biomol. Chem. 11, 2241-2244 (2013).
22. Chandra, M. & Silverman, S. K. DNA and RNA Can Be Equally Efficient Catalysts for Carbon-Carbon Bond Formation. J. Am. Chem. Soc. 130, 2936-2937 (2008).
23. Li, Y. & Sen, D. Toward an Efficient DNAzyme. Biochemistry 36, 5589-5599 (1997).
24. Travascio, P., Li, Y. & Sen, D. DNA-enhanced peroxidase activity of a DNA aptamer-hemin complex. Chemistry & Biology 5, 505-517 (1998).
25. Yao, T. et al. DNAzymes for amine and peptide lysine acylation. Org. Biomol. Chem. 19, 171-181 (2021).
26. Brandsen, B. M., Hesser, A. R., Castner, M. A., Chandra, M. & Silverman, S. K. DNA-Catalyzed Hydrolysis of Esters and Aromatic Amides. J. Am. Chem. Soc. 135, 16014-16017 (2013).
27. Ponce-Salvatierra, A., Boccaletto, P. & Bujnicki, J. M. DNAmoreDB, a database of DNAzymes. Nucleic Acids Research 49, D76-D81 (2021).
28. Liu, K., Lat, P. K., Yu, H.-Z. & Sen, D. CLICK-17, a DNA enzyme that harnesses ultra-low concentrations of either Cu+ or Cu2+ to catalyze the azide-alkyne ‘click’ reaction in water. Nucleic Acids Research 48, 7356-7370 (2020).
29. Kanan, M. W., Rozenman, M. M., Sakurai, K., Snyder, T. M. & Liu, D. R. Reaction discovery enabled by DNA-templated synthesis and in vitro selection. Nature 431, 545-549 (2004).
30. Chen, Y., Kamlet, A. S., Steinman, J. B. & Liu, D. R. A Biomolecule-Compatible Visible Light-Induced Azide Reduction from a DNA-Encoded Reaction Discovery System. Nat Chem 3, 146-153 (2011).
31. Hook, K. D., Chambers, J. T. & Hili, R. A platform for high-throughput screening of DNA-encoded catalyst libraries in organic solvents. Chem. Sci. 8, 7072-7076 (2017).
32. Hoover, J. M. & Stahl, S. S. Highly Practical Copper(I)/TEMPO Catalyst System for Chemoselective Aerobic Oxidation of Primary Alcohols. J. Am. Chem. Soc. 133, 16901-16910 (2011).
33. Hoover, J. M., Ryland, B. L. & Stahl, S. S. Mechanism of Copper(I)/TEMPO-Catalyzed Aerobic Alcohol Oxidation. J. Am. Chem. Soc. 135, 2357-2367 (2013).
34. Hansen, M. H. et al. A Yoctoliter-Scale DNA Reactor for Small-Molecule Evolution. J. Am. Chem. Soc. 131, 1322-1327 (2009).
35. Halpin, D. R. & Harbury, P. B. DNA Display I. Sequence-Encoded Routing of DNA Populations. PLOS Biology 2, e173 (2004).
36. Lilley, D. M. J. Structures of helical junctions in nucleic acids. Quarterly Reviews of Biophysics 33, 109-159 (2000).
37. Ahneman, D. T., Estrada, J. G., Lin, S., Dreher, S. D. & Doyle, A. G. Predicting reaction performance in C—N cross-coupling using machine learning. Science 360, 186 (2018).
38. Gartner, Z. J. & Liu, D. R. The Generality of DNA-Templated Synthesis as a Basis for Evolving Non-Natural Small Molecules. J. Am. Chem. Soc. 123, 6961-6963 (2001).
39. Zultanski, S. L., Zhao, J. & Stahl, S. S. Practical Synthesis of Amides via Copper/ABNO-Catalyzed Aerobic Oxidative Coupling of Alcohols and Amines. J. Am. Chem. Soc. 138, 6416-6419 (2016).
40. Hungerhoff, B., Samanta, S. S., Roels, J. & Metz, P. Chemoselective Oxidative Debenzylation of N,N-Dibenzylamines. Synlett 2000, 77-79 (2000).
41. Bull, S. D. et al. Chemoselective debenzylation of N-benzyl tertiary amines with ceric ammonium nitrate. J. Chem. Soc., Perkin Trans. 1 3765-3774 (2000) doi: 10.1039/B006852G.
42. Müller, P. & Gilabert, D. M. Oxidation of amines to imines with hypervalent iodine. Tetrahedron 44, 7171-7175 (1988).
43. Choi, H. & Doyle, M. P. Oxidation of secondary amines catalyzed by dirhodium caprolactamate. Chem. Commun. 745-747 (2007) doi: 10.1039/B615805F.
44. Chu, G. & Li, C. Convenient and clean synthesis of imines from primary benzylamines. Org. Biomol. Chem. 8, 4716-4719 (2010).
45. Yamazaki, S. Oxidation of Secondary Amines with NiSO4-K2S2O8. Chem. Lett. 21, 823-826 (1992).
46. Grayson, E. J. & Davis, B. G. A Tuneable Method for N-Debenzylation of Benzylamino Alcohols. Org. Lett. 7,2361-2364 (2005).
47. Gore, G. M. et al. Synthesis of CL-20 (IV) by Oxidative Debenzylation with Cerium(IV) Ammonium Nitrate (CAN). ChemInform 38, (2007).
48. Schümperli, M. T., Hammond, C. & Hermans, I. Developments in the Aerobic Oxidation of Amines. ACS Catal. 2, 1108-1117 (2012).
49. Patil, R. D. & Adimurthy, S. Catalytic Methods for Imine Synthesis. Asian Journal of Organic Chemistry 2, 726-744 (2013).
50. Patil, R. D. & Adimurthy, S. Copper-Catalyzed Aerobic Oxidation of Amines to Imines under Neat Conditions with Low Catalyst Loading. Advanced Synthesis & Catalysis 353, 1695-1700 (2011).
51. Maeda, Y., Nishimura, T. & Uemura, S. Copper-Catalyzed Oxidation of Amines with Molecular Oxygen. BCSJ 76, 2399-2403 (2003).
52. Éll, A. H., Samec, J. S. M., Brasse, C. & Bäckvall, J.-E. Dehydrogenation of aromatic amines to imines via ruthenium-catalyzed hydrogen transfer. Chem. Commun. 1144-1145 (2002) doi: 10.1039/B202117J.
53. Samec, J. S. M., Éll, A. H. & Bäckvall, J.-E. Efficient Ruthenium-Catalyzed Aerobic Oxidation of Amines by Using a Biomimetic Coupled Catalytic System. Chemistry—A European Journal 11, 2327-2334 (2005).
54. Dhakshinamoorthy, A., Alvaro, M. & Garcia, H. Aerobic Oxidation of Benzyl Amines to Benzyl Imines Catalyzed by Metal-Organic Framework Solids. ChemCatChem 2, 1438-1443 (2010).
55. Jing, Y. et al. Activation of Dioxygen by Cobaloxime and Nitric Oxide for Efficient TEMPO-Catalyzed Oxidation of Alcohols. Advanced Synthesis & Catalysis 353, 1146-1152 (2011).
56. Sonobe, T., Oisaki, K. & Kanai, M. Catalytic aerobic production of imines en route to mild, green, and concise derivatizations of amines. Chem. Sci. 3, 3249-3255 (2012).
57. Hu, Z. & Kerton, F. M. Simple copper/TEMPO catalyzed aerobic dehydrogenation of benzylic amines and anilines. Org. Biomol. Chem. 10, 1618-1624 (2012).
58. Huang, B. et al. Cu(I)/TEMPO-catalyzed aerobic oxidative synthesis of imines directly from primary and secondary amines under ambient and neat conditions. Tetrahedron Letters 54, 2861-2864 (2013).
59. Ray, R. et al. Ligand controlled switchable selectivity in ruthenium catalyzed aerobic oxidation of primary amines. Chem. Commun. 53, 4006-4009 (2017).
60. Nishinaga, A., Yamazaki, S. & Matsuura, T. Catalytic dehydrogenation of secondary amines with cobalt schiff base complex-oxygen system. Tetrahedron Letters 29, 4115-4118 (1988).
61. Zultanski, S. L., Zhao, J. & Stahl, S. S. Practical Synthesis of Amides via Copper/ABNO-Catalyzed Aerobic Oxidative Coupling of Alcohols and Amines. J. Am. Chem. Soc. 138, 6416-6419 (2016).
62. Gellrich, U., Khusnutdinova, J. R., Leitus, G. M. & Milstein, D. Mechanistic Investigations of the Catalytic Formation of Lactams from Amines and Water with Liberation of H2. J Am. Chem. Soc. 137, 4851-4859 (2015).
63. Tang, S. et al. Catalytic Oxidative Deamination by Water with H2 Liberation. J Am Chem Soc 142, 20875-20882 (2020).
64. Urgoitia, G., SanMartin, R., Herrero, M. T. & Dominguez, E. Vanadium-Catalyzed Oxidative Debenzylation of O-Benzyl Ethers at ppm Level. Advanced Synthesis & Catalysis 358, 3307-3312 (2016).
65. Urgoitia, G., SanMartin, R., Herrero, M. T. & Domínguez, E. Optimization of process conditions for nickel-catalyzed selective aerobic O-debenzylation. Applied Catalysis A: General 579, 86-90 (2019).
66. Hruszkewycz, D. P., Miles, K. C., Thiel, O. R. & Stahl, S. S. Co/NHPI-mediated aerobic oxygenation of benzylic C—H bonds in pharmaceutically relevant molecules. Chem. Sci. 8, 1282-1287 (2017).
67. Wang, Y., DuBois, J. L., Hedman, B., Hodgson, K. O. & Stack, T. D. P. Catalytic Galactose Oxidase Models: Biomimetic Cu(II)-Phenoxyl-Radical Reactivity. Science 279, 537-540 (1998).
68. Minakata, S. et al. Catalytic Oxidation of Amines Utilizing Binuclear Copper(II) Complex of 7-Azaindole. Chem. Lett. 26, 311-312 (1997).
69. L. Jain, S. & Sain, B. Ruthenium catalyzed oxidation of tertiary nitrogen compounds with molecular oxygen: an easy access to N-oxides under mild conditions. Chemical Communications 0, 1040-1041 (2002).
70. Xu, B., Lumb, J.-P. & Arndtsen, B. A. A TEMPO-Free Copper-Catalyzed Aerobic Oxidation of Alcohols. Angewandte Chemie International Edition 54, 4208-4211 (2015).
71. McCann, S. D., Lumb, J.-P., Arndtsen, B. A. & Stahl, S. S. Second-Order Biomimicry: In Situ Oxidative Self-Processing Converts Copper(I)/Diamine Precursor into a Highly Active Aerobic Oxidation Catalyst. ACS Cent. Sci. 3, 314-321 (2017).
72. Hoover, J. M., Ryland, B. L. & Stahl, S. S. Mechanism of Copper(I)/TEMPO-Catalyzed Aerobic Alcohol Oxidation. J. Am. Chem. Soc. 135, 2357-2367 (2013).
73. Murahashi, S.-I., Naota, T. & Taki, H. Ruthenium-catalysed oxidation of secondary amines to imines using t-butyl hydroperoxide. J. Chem. Soc., Chem. Commun. 613-614 (1985) doi: 10.1039/C39850000613.
74. Mairal Lerga, T. et al. High Affinity Aptamer for the Detection of the Biogenic Amine Histamine. Anal. Chem. 91, 7104-7111 (2019).
75. Pandey, G. & Rani, K. S. Photosensitized single electron transfer initiated N-debenzylation. A convenient and mild approach. Tetrahedron Letters 29, 4157-4158 (1988).
76. Narayanam, J. M. R., Tucker, J. W. & Stephenson, C. R. J. Electron-Transfer Photoredox Catalysis: Development of a Tin-Free Reductive Dehalogenation Reaction. J. Am. Chem. Soc. 131, 8756-8757 (2009).
77. Liu, Z., Zhang, Y., Cai, Z., Sun, H. & Cheng, X. Photoredox Removal of p-Methoxybenzyl Ether Protecting Group with Hydrogen Peroxide as Terminal Oxidant. Advanced Synthesis & Catalysis 357, 589-593 (2015).
78. Ahn, D. K., Kang, Y. W. & Woo, S. K. Oxidative Deprotection of p-Methoxybenzyl Ethers via Metal-Free Photoredox Catalysis. J. Org. Chem. 84, 3612-3623 (2019).
79. Lardy, S. & Schmidt, V. Thiol Catalyzed Aerobic Debenzylation of Alcohols and Amines. (2021) doi: 10.33774/chemrxiv-2021-w3 q1d.
80. Zuo, Z. et al. Merging photoredox with nickel catalysis: Coupling of α-carboxyl sp3-carbons with aryl halides. Science 345, 437-440 (2014).
81. Tellis, J. C., Primer, D. N. & Molander, G. A. Single-electron transmetalation in organoboron cross-coupling by photoredox/nickel dual catalysis. Science 345, 433-436 (2014).
82. Corcoran, E. B. et al. Aryl amination using ligand-free Ni(II) salts and photoredox catalysis. Science 353, 279-283 (2016).
83. Diccianni, J. B. & Diao, T. Mechanisms of Nickel-Catalyzed Cross-Coupling Reactions. Trends in Chemistry 1, 830-844 (2019).
84. Phelan, J. P. et al. Open-Air Alkylation Reactions in Photoredox-Catalyzed DNA-Encoded Library Synthesis. J. Am. Chem. Soc. 141, 3723-3732 (2019).
85. Geri, J. B. et al. Microenvironment mapping via Dexter energy transfer on immune cells. Science 367, 1091 (2020).
86. Zheng, S., Gutierrez-Bonet, A. & Molander, G. A. Merging Photoredox PCET with Ni-Catalyzed Cross-Coupling: Cascade Amidoarylation of Unactivated Olefins. Chem 5, 339-352 (2019).
87. Pan, Y. et al. Boosting Photocatalytic Activities for Organic Transformations through Merging Photocatalyst and Transition-Metal Catalyst in Flexible Polymers. ACS Catal. 10, 11758-11767 (2020).
88. Ahn, J. M., Peters, J. C. & Fu, G. C. Design of a Photoredox Catalyst that Enables the Direct Synthesis of Carbamate-Protected Primary Amines via Photoinduced, Copper-Catalyzed N-Alkylation Reactions of Unactivated Secondary Halides. J. Am. Chem. Soc. 139, 18101-18106 (2017).
89. Musacchio Andrew J. et al. Catalytic intermolecular hydroaminations of unactivated olefins with secondary alkyl amines. Science 355, 727-730 (2017).

### Example 2

This Example further demonstrates that the platform system can be used for library development and screening.

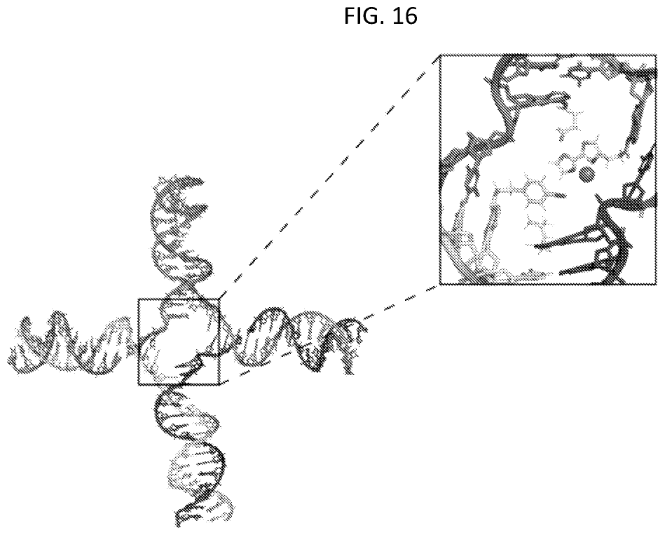

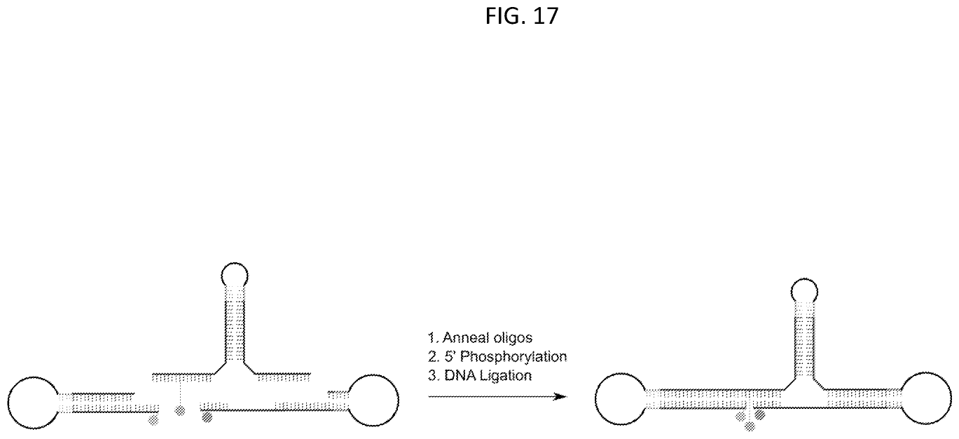

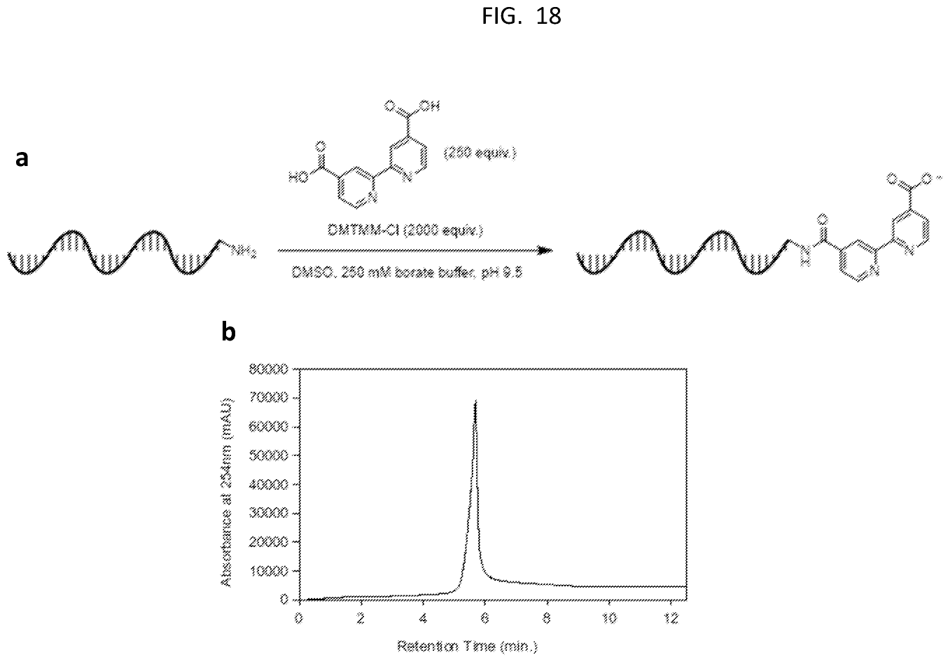

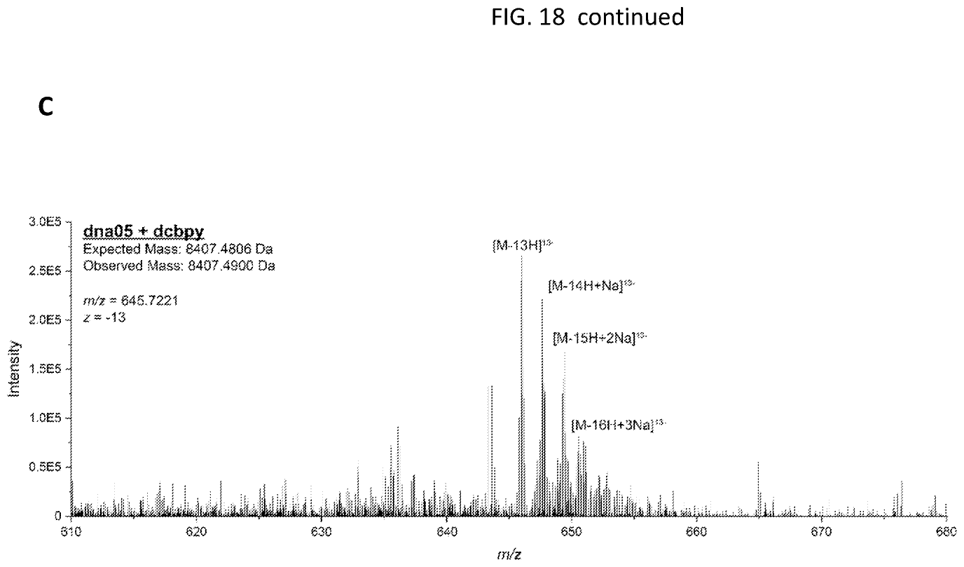

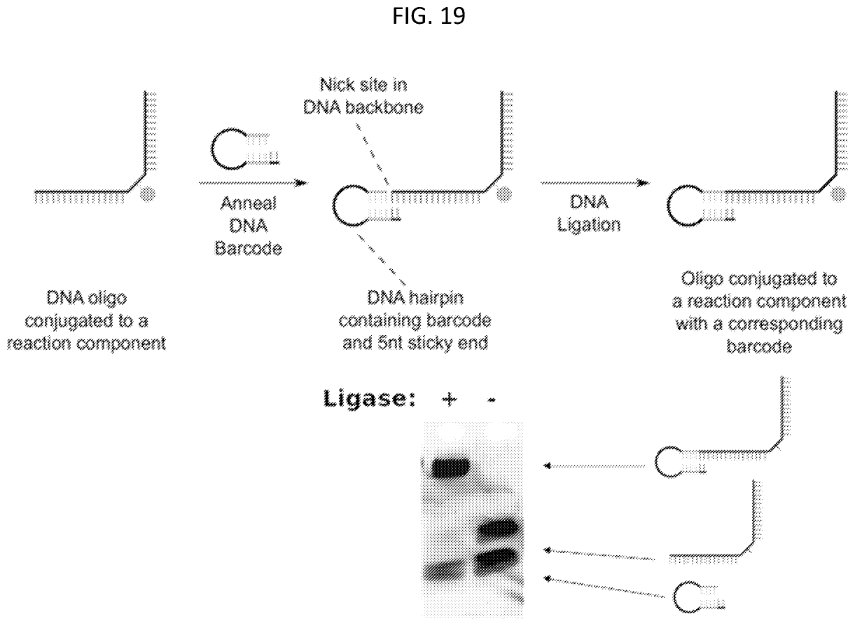

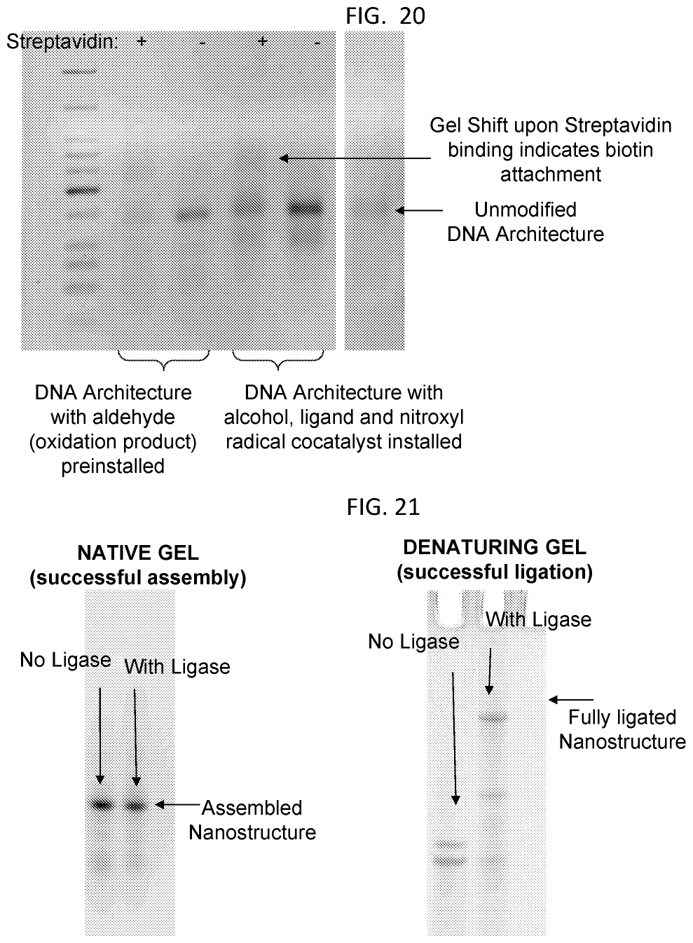

A model of a polynucleotide nanoscaffold with a detailed view of the catalytic active site (inset), is shown with regard to a four polynucleotide barcoded building block oligomers that form a self-assembled polynucleotide scaffold (FIG. 16). The DNA can self-assemble into hairpin structures forming a middle cavity in which the catalytic components can be tethered as to bring into close proximity. A schematic illustration of the initial architecture which allows for combinatorial screening of three components is depicted if FIG. 17. Barcoding regions of the polynucleotide sequence are correlated to each unique catalytic component in the assembled nanoscaffold and are depicted in the same color to coordinate the catalytic component and barcode. The two larger hairpins on the left and right are the primer binding sites for DNA amplification and later next generation sequencing. Attachment of the ligand and organic radical co-catalyst is demonstrated in FIG. 18. Panel A shows a synthetic scheme for bioconjugation that was confirmed by HPLC and ESI-MS. Panel B shows a high-performance liquid chromatogram and panel C shows an electrospray ionization mass spectra confirming the synthetic method. This method has been demonstrated with additional ligands including proline, alternative bipyridine ligands, various alcohol substrates, alkynes, phosphines, aromatic and alkyl groups, and organometallic complexes (data not shown). Thus, this demonstrates that the single stranded polynucleotides can be tethered to the catalyst components. A schematic of successful barcode attachment for identifying individual catalytic components is shown in FIG. 19. The attachment of hairpin barcodes proceeds quantitatively. In the gel shown there was a slight excess of barcoding hairpin. Further confirmation is shown in FIG. 20. where an electrophoretic gel demonstrates successful oxidation and reductive amination to attach biotin for isolating successful catalytic combinations. The gel shows that biotin attachment to an aldehyde leads to a gel shift. When all of the reaction components are present, oxidation if the alcohol creates an aldehyde in situ and biotin-PEG7-NH2 can be attached through reductive amination. When one or more components are missing, no shift is observed (not shown).

The assembly and ligation of nanostructures is further demonstrated in FIG. 21. Electrophoretic gels showing the assembly and ligation of polynucleotide nanoscaffolds. High-yielding assembly is observed in the native gel and is also demonstrated with DNA oligos with catalytic components attached. Denaturing gels shows that the nanostructure is successfully ligated. Downstream PCR is also successful.

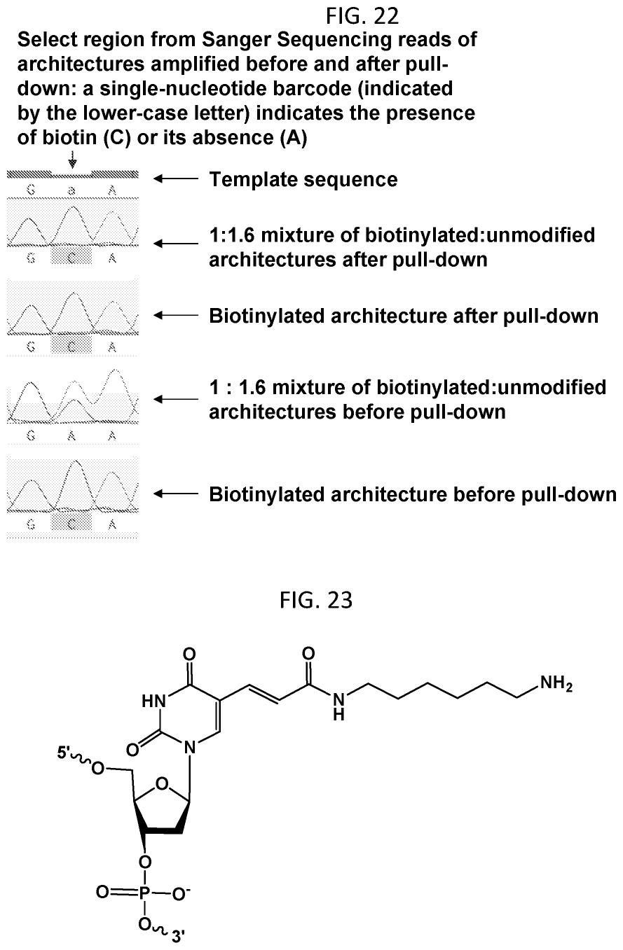

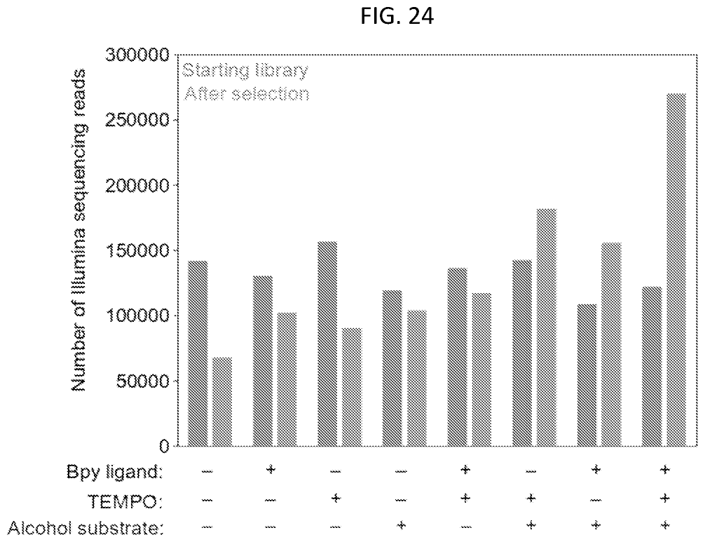

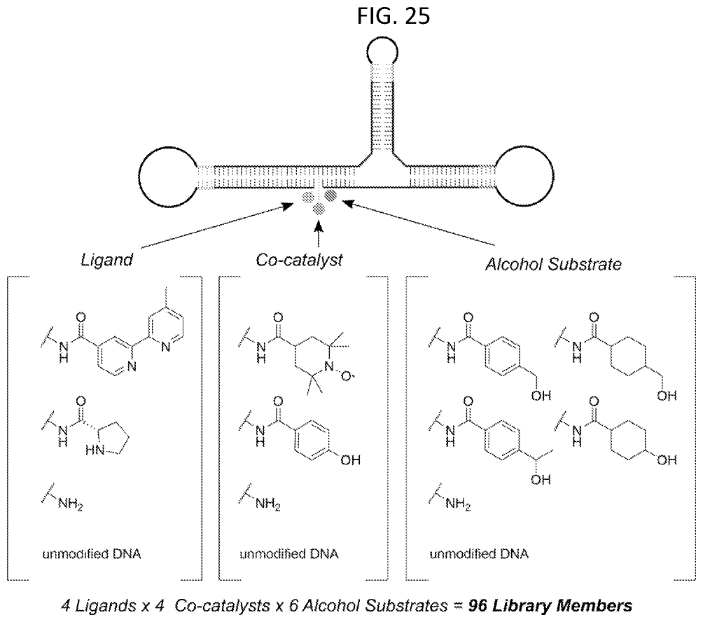

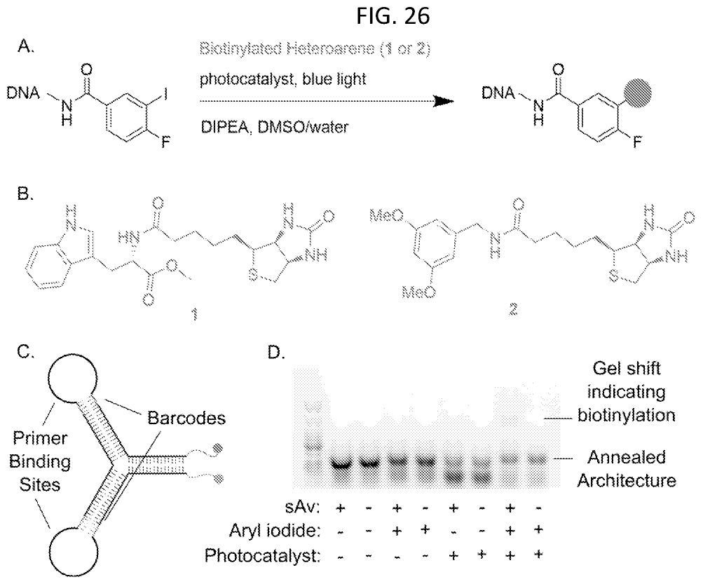

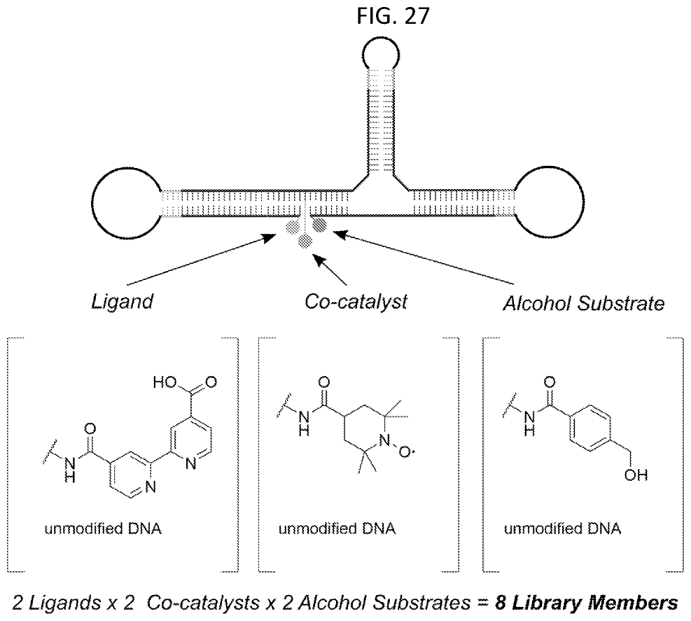

Further, we have demonstrated that pull-down enriches for the biotin-tagged structures, demonstrated in FIG. 22. Streptavidin pull-down can be used to enrich biotin-tagged structures. Mixtures of polynucleotide structures with and without biotin (with barcodes to indicate the presence or absence of the biotin modification) were exposed to magnetic streptavidin beads. Biotinylated structures bound tightly, while unmodified structures were washed away. Amplification of the sequences before and after pull-down followed by sequencing shows that only the biotin barcode is identified after pull-down.

Lastly, FIG. 23 demonstrates that the PCR protocol used in polymerase chain reaction (PCR) amplification tolerates catalytic components covalently linked to the DNA nanostructure being PCR amplified. For the internal attachment site, we used a commercially available attachment handle (internal amine C6 dT modification, from IDT) which is tolerated by Taq polymerases. We have demonstrated successful amplification of nanostructures bearing multiple reaction components.

The Sanger sequencing for an initial, small proof-of-principle library (<30 library members) demonstrated that single-nucleotide barcodes and Sanger Sequencing are sufficient to confirm that the expected structures are enriched by the pull-down, but larger libraries will require at least 5 nucleotide barcodes to be used.

### Example 3

### Synthetic Schemes

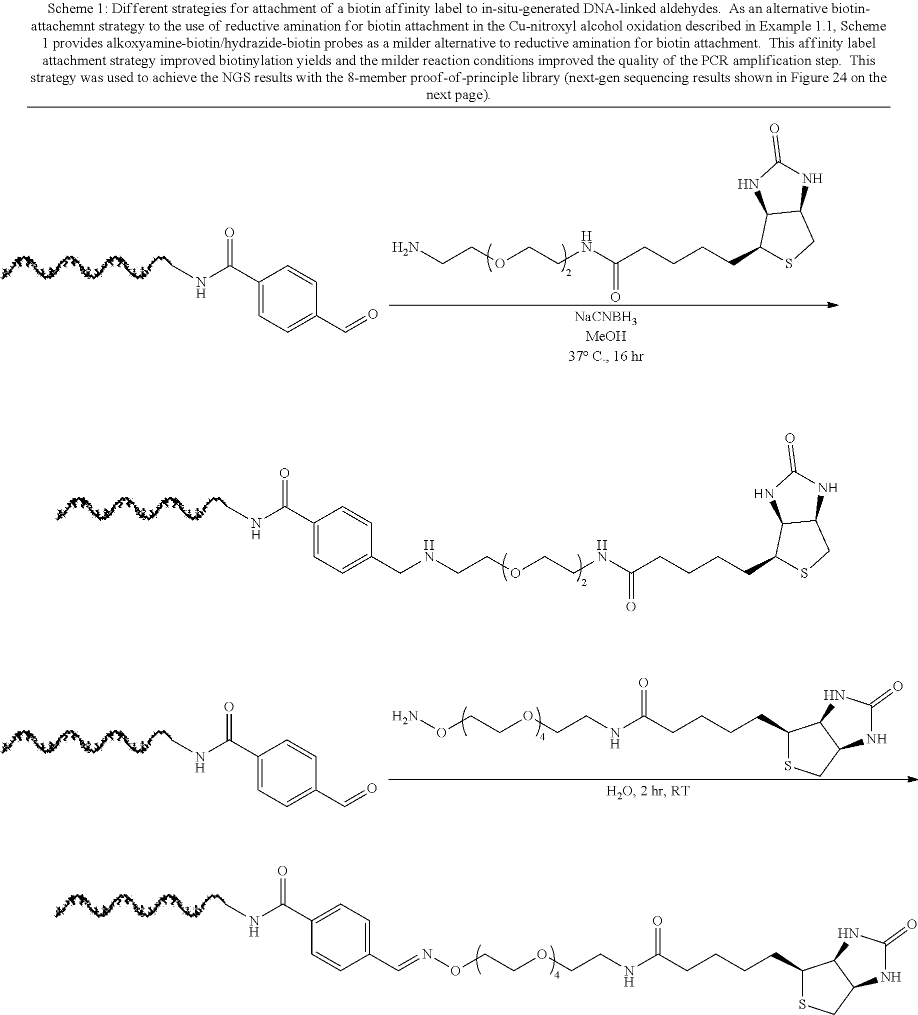

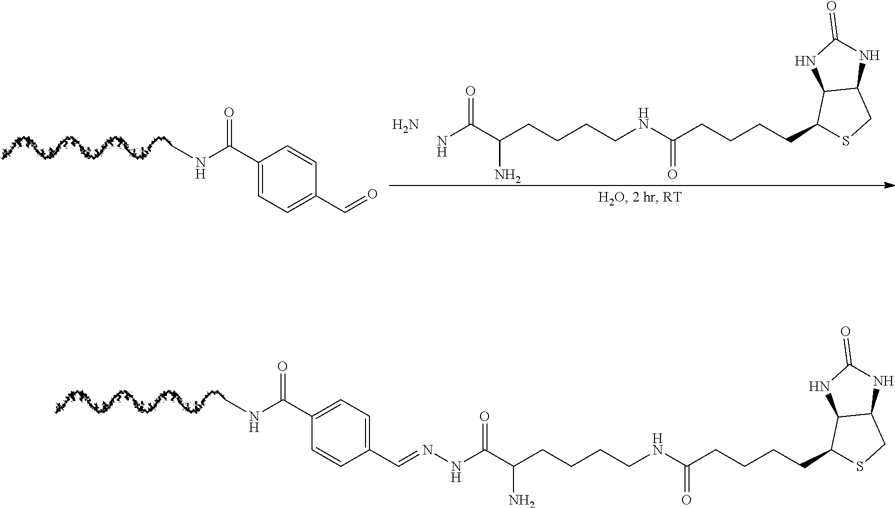

### Amide-Bond-Forming Bioconjugation Protocols for Preparation of Library Building Blocks.

These following schemes and procedures are for DMTMM and EDC couplings to attach carboxylic acid-bearing reaction components to amine-modified DNA oligonucleotides.

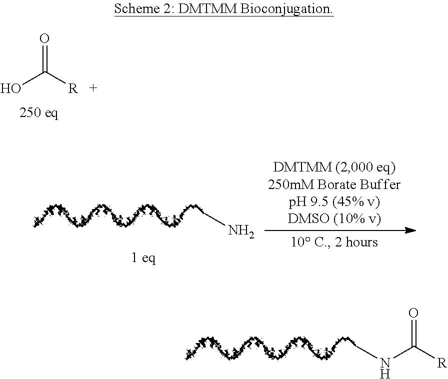

To 1 nmol amine-modified DNA (1 mM, 1 μL in nuclease-free water), 9 μL 250 mM Borate Buffer pH 9.5 was added. Following this addition 250 equivalents of the carboxylic acid were added (0.125 M in DMSO, 2 μL) as well as 2 μL DMSO. Last, 2,000 equivalents of DMTMM (4-(4,6-Dimethoxy-1,3,5-triazin-2-yl)-4-methylmorpholinium chloride) were added (4 μL, 0.5 M in nuclease-free water). The reaction was vortexed and incubated at 10° C. with vigorous shaking for 2 hours. The DNA was then purified via isopropanol precipitation (precipitate with 3 M sodium acetate and cold isopropanol, wash with 70% ethanol), or via filtration with a molecular weight cut-off filter (3 kDa cut-off).

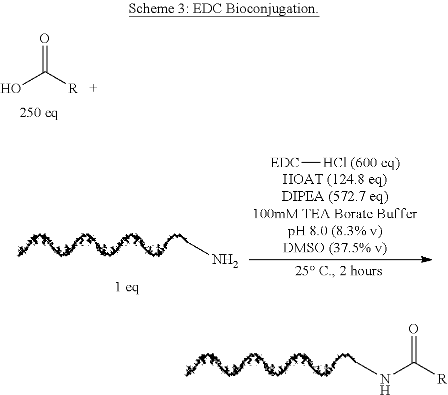

To 1 nmol amine-modified DNA (1 mM, 1 μL in nuclease-free water), 2 μL 100 mM TEA Borate Buffer pH 8.0 was added. Following this addition, 250 equivalents of the carboxylic acid were added (0.125 M in DMSO, 2 μL) as well as 9 μL DMSO. Last, 600 equivalents of EDC-HCl (N-(3-Dimethylaminopropyl)-N′-ethylcarbodiimide hydrochloride) (4 μL, 0.15 M in DMSO), 124.8 equivalents of HOAt (1-Hydroxy-7-azabenzotriazole) (4 μL, 0.03 M in DMSO), and 572.7 equivalents of DIPEA (N,N-Diisopropylethylamine) (2 μL, 0.286 M) were added. The reaction was vortexed and incubated at 25° C. for 2 hours. The DNA was then purified via isopropanol Precipitation (precipitate with 3 M sodium acetate and cold isopropanol, wash with 70% ethanol), or via filtration with a molecular weight cut-off filter (3 kDa cut-off).

For the photochemical C—H arylation reaction, two possible biotinylated coupling partners, that were not commercially available, were synthesized. The biotin-hydrazide, biotin-amine, and biotin-alkoxyamine probes used for aldehyde labelling in the Cu/Nitroxyl Radical catalysis experiments are all commercially available.

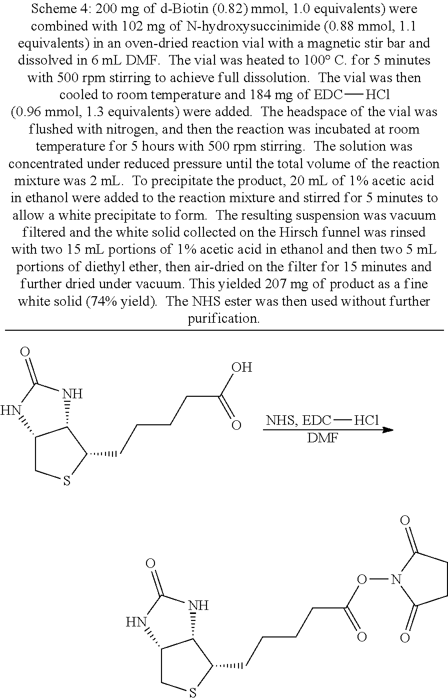

¹H NMR (400 MHz, DMSO) δ 6.42 (s, 1H), 6.36 (s, 1H), 4.30 (dd, J=7.7, 5.0 Hz, 1H), 4.18-4.10 (m, 1H), 3.15-3.06 (m, 1H), 2.91-2.80 (m, 1H), 2.81 (s, 4H), 2.67 (t, J=7.4 Hz, 2H), 2.58 (d, J=12.4 Hz, 1H), 1.73-1.33 (m, 6H).

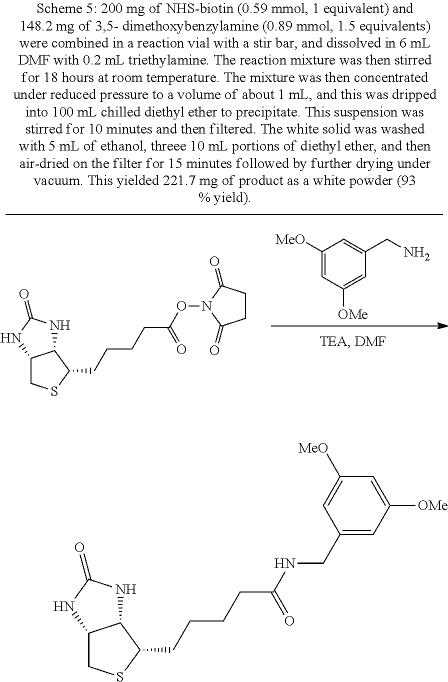 [SMILES: O=C1N[C@H]2[C@H](CS[C@H]2CCCCC(=O)ON2C(=O)CCC2=O)N1 ; COc1cc(CN)cc(OC)c1 ; COc1cc(CNC(=O)CCCC[C@@H]2SC[C@@H]3NC(=O)N[C@@H]32)cc(OC)c1]

¹H NMR (500 MHz, DMSO) δ 8.25 (t, J=6.0 Hz, 1H), 6.41 (d, J=1.7 Hz, 1H), 6.40 (d, J=2.3 Hz, 2H), 6.35 (t, J=2.3 Hz, 1H), 6.34 (s, 1H), 4.30 (ddt, J=7.7, 5.3, 1.1 Hz, 1H), 4.19 (d, J=6.0 Hz, 2H), 4.12 (ddd, J=7.7, 4.4, 1.9 Hz, 1H), 3.71 (s, 6H), 3.09 (ddd, J=8.6, 6.2, 4.4 Hz, 1H), 2.82 (dd, J=12.4, 5.1 Hz, 1H), 2.58 (d, J=12.4 Hz, 1H), 2.14 (t, J=7.4 Hz, 2H), 1.70 -1.22 (m, 6H).

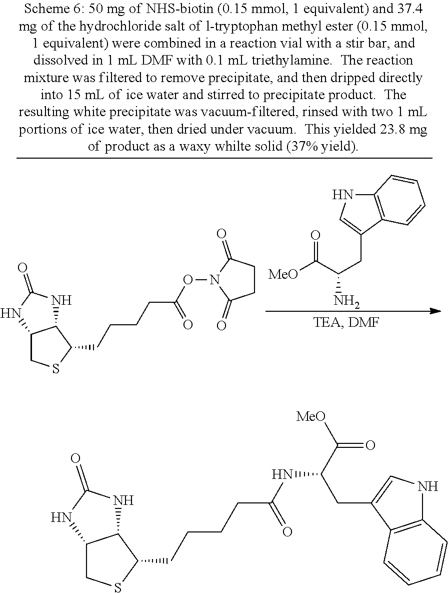 [SMILES: O=C1N[C@H]2[C@H](CS[C@H]2CCCCC(=O)ON2C(=O)CCC2=O)N1 ; COC(=O)[C@@H](N)Cc1c[nH]c2ccccc12 ; COC(=O)[C@H](Cc1c[nH]c2ccccc12)NC(=O)CCCC[C@@H]1SC[C@@H]2NC(=O)N[C@@H]21]

¹H NMR (500 MHz, DMSO) δ 10.90-10.79 (m, 1H), 8.21 (d, J=7.6 Hz, 1H), 7.48 (dd, J=7.9, 1.0 Hz, 1H), 7.33 (dt, J=8.1, 0.9 Hz, 1H), 7.13 (d, J=2.4 Hz, 1H), 7.06 (ddd, J=8.1, 6.9, 1.2 Hz, 1H), 6.98 (ddd, J=7.9, 7.0, 1.1 Hz, 1H), 6.40-6.29 (m, 2H), 4.51 (td, J=8.2, 5.8 Hz, 1H), 4.33-4.26 (m, 1H), 4.09 (ddd, J=7.7, 4.4, 1.9 Hz, 1H), 3.58 (s, 3H), 3.14 (dd, J=14.6, 5.6 Hz, 1H), 3.08-2.97 (m, 2H), 2.82 (dd, J=12.4, 5.1 Hz, 1H), 2.57 (d, J=12.4 Hz, 1H), 2.16-2.00 (m, 2H), 1.68-1.13 (m, 6H).

1. A polynucleotide barcoded building block oligomer system for preparing a catalyst library system, the polynucleotide system comprising at least two sets of single stranded polynucleotides, each set of single stranded polynucleotides characterized by a catalytic component selected from a panel of catalytic components linked to single stranded polynucleotides of the set,
wherein each single stranded polynucleotide of a set comprises a polynucleotide barcode indicative of the catalytic component selected from the panel of catalytic components linked to the single stranded polynucleotide, a domain complementary to a domain possessed by single stranded polynucleotides of a second set of single stranded polynucleotides and optionally a domain complementary to a domain possessed by single stranded polynucleotides of a third set of single stranded polynucleotides,
wherein each single stranded polynucleotide of one set is capable of hybridizing with each single stranded polynucleotide of at least one other set to form a self-assembled polynucleotide nanoscaffold, and
wherein the self-assembled polynucleotide nanoscaffold comprises a catalytic active site comprising the catalytic components and a barcode signature indicative of the catalytic active site.

2. The polynucleotide system of claim 1, wherein one set of single stranded polynucleotides comprises a catalytic component selected from a panel of catalysts or catalyst binding ligands and another set of single stranded polynucleotides comprises a catalytic component selected from a panel of substrates.

3. The polynucleotide barcoded building block oligomer system of claim 1 comprising 3, 4, or 5 sets of single stranded polynucleotides.

4. The polynucleotide barcoded building block oligomer system of claim 3, wherein a first set of single stranded polynucleotides comprises a catalytic component selected from a panel of catalysts or catalyst binding ligands, a second set of single stranded polynucleotides comprises a catalytic component selected from a panel of substrates, and a third set of single stranded polynucleotides comprises a catalytic component selected from a panel of co-catalysts, additives, acids, bases, H-donors, H-acceptors, aptamers, or any combination thereof.

5. The polynucleotide barcoded building block oligomer system of claim 1, wherein the catalytic component is linked to the single stranded polynucleotide by carboxylic acid-amine bioconjugation.

6. The polynucleotide barcoded building block oligomer system for preparing a catalyst library system of claim 1,
wherein each single stranded polynucleotide of the first set comprises a first polynucleotide barcode indicative of a first catalytic component selected from a first panel of L catalytic components linked to the single stranded polynucleotide and a domain complementary to a domain possessed by single stranded polynucleotides of a second set of single stranded polynucleotides,
wherein each single stranded polynucleotide of the second set comprises a second polynucleotide barcode indicative of a second catalytic component selected from a second panel of M catalytic components linked to the single stranded polynucleotides of the second set, and
wherein each single stranded polynucleotide of the first set and each single stranded polynucleotide of the second set are capable of hybridizing with each other to form a self-assembled polynucleotide nanoscaffold, and
wherein the self-assembled polynucleotide nanoscaffold comprises a catalytic active site comprising the first catalytic component and the second catalytic component and a barcode signature indicative of the catalytic active site comprising the first polynucleotide barcode and the second polynucleotide barcode.

7. The polynucleotide barcoded building block oligomer system of claim 6,
wherein each single stranded polynucleotide of the second set comprises a domain complementary to a domain possessed by single stranded polynucleotides of a third set of single stranded polynucleotides,
wherein each single stranded polynucleotide of the third set comprises a third polynucleotide barcode indicative of a third catalytic component selected from a third panel of N catalytic components linked to the single stranded polynucleotides of the third set, and
wherein each single stranded polynucleotide of the first set, each single stranded polynucleotide of the second set, and each single stranded polynucleotide of the third set, are capable of forming a self-assembled polynucleotide nanoscaffold, and
wherein the self-assembled polynucleotide nanoscaffold comprises a catalytic active site comprising the first catalytic component, the second catalytic component, and the third catalytic component and a barcode signature indicative of the catalytic active site comprising the first polynucleotide barcode, the second polynucleotide barcode, and the third polynucleotide barcode.

8. A catalyst system library comprising a plurality of self-assembled polynucleotide nanoscaffolds prepared from polynucleotide barcoded building block oligomer system according to claim 1.

9. The catalyst system library of claim 8, wherein the self-assembled polynucleotide nanoscaffolds comprise a single stranded polynucleotide selected from each set of single stranded polynucleotides.

10. The catalyst system library of claim 9, wherein the self-assembled polynucleotide nanoscaffolds comprise a continuous polynucleotide sequence.

11. The catalyst system library of claim 10, wherein the self-assembled polynucleotide nanoscaffolds comprise at least one hairpin structure.

12. The catalyst system library of claim 8, wherein the self-assembled polynucleotide nanoscaffolds comprise a reporter attached to the polynucleotide nanoscaffold.

13. The catalyst system library of claim 12, wherein the reporter is biotin, optionally wherein the biotin is conjugated to the polynucleotide nanoscaffold by alkoxyamine-biotin or hydrazide-biotin.

14. A method of assembling a catalyst system library, the method comprising:
preparing a polynucleotide barcoded building block oligomer system according to claim 1, wherein a set of single stranded polynucleotides is prepared by
(i) distributing a first single stranded polynucleotide comprising a domain complementary to a domain possessed by a second single stranded polynucleotide and optionally a domain complementary to a domain possessed by a third stranded polynucleotide between a set of containers,
(ii) adding to each container of the set of containers a catalytic component selected from a panel of catalytic components and a polynucleotide barcode indicative of the catalytic component selected from the panel,
(iii) attaching to the single stranded polynucleotide the catalytic component selected from a panel of catalytic components, and
(iv) ligating the single stranded polynucleotide and the polynucleotide barcode,
combining the two or more sets of single stranded polynucleotides under conditions sufficient to prepare self-assembled polynucleotide nanoscaffolds; and
ligating the self-assembled polynucleotide nanoscaffolds to prepare a continuous polynucleotide sequence.

15. A method of identifying catalytic activity, the method comprising exposing the catalyst system library according to claim 8 to catalytic reaction conditions and identifying self-assembled polynucleotide nanoscaffolds that react under the catalytic reaction conditions.

16. The method of claim 15, wherein identifying self-assembled polynucleotide nanoscaffold that react under the catalytic reaction conditions comprises,
isolating self-assembled polynucleotide nanoscaffolds with catalytic activity,
amplifying a portion of the self-assembled polynucleotide nanoscaffolds comprising the barcode signature, and
sequencing the portion of the self-assembled polynucleotide nanoscaffolds to determine the barcode signature.

17. The method of claim 16, wherein isolating the self-assembled polynucleotide nanoscaffolds comprises isolating the self-assembled polynucleotide nanoscaffolds comprising a reporter, wherein self-assembled polynucleotide nanoscaffolds comprising the reporter is associated with catalytic activity.

18. The method of claim 16, wherein isolating the self-assembled polynucleotide nanoscaffolds comprises isolating the self-assembled polynucleotide nanoscaffolds lacking a reporter, wherein self-assembled polynucleotide nanoscaffolds lacking the reporter from the scaffold indicates catalytic activity.

19. The method of claim 16, wherein the portion of the self-assembled polynucleotide nanoscaffolds comprising the barcode signature is amplified by a polymerase chain reaction.

20. The method of claim 20, wherein the portion of the self-assembled polynucleotide nanoscaffolds comprising the barcode signature is sequenced by next generation polynucleotide sequencing.
